(19) United States
(12) Patent Application Publication
(10) Pub. No.: US 2025/0041442 A1
(43) Pub. Date: Feb. 6, 2025

(54) DIAMINO LIPID (DAL) COMPOUNDS AND PHARMACEUTICAL COMPOSITIONS COMPRISING AN IMMUNOTHERAPEUTIC AGENT

(71) Applicant: Ohio State Innovation Foundation, Columbus, OH (US)

(72) Inventors: Yizhou Dong, New York, NY (US); Xue-Feng BAI, Columbus, OH (US); Jin-Qing LIU, Columbus, OH (US); Chengxiang ZHANG, Cambridge, MA (US); Xinfu ZHANG, Dalian (CN)

(21) Appl. No.: 18/716,721

(22) PCT Filed: Dec. 5, 2022
(86) PCT No.: PCT/US2022/051861

§ 371 Date: Jun. 5, 2024

Related U.S. Application Data
(60) Provisional application No. 63/286,272, filed on Dec. 6, 2021.

(51) Int. Cl. A61K 48/00; A61K 9/51; A61K 38/19; A61K 38/20; A61P 35/00; C07C 237/06; C07F 5/02

(52) U.S. Cl. CPC A61K 48/0033; A61K 9/5123; A61K 38/193; A61K 38/20; A61K 38/208; A61P 35/00; C07C 237/06; C07F 5/025; C07C 2601/16

(57) ABSTRACT

Disclosed herein are diamino lipid (dal) compounds and pharmaceutical compositions including mRNA encoding an immunotherapeutic agent and methods of making and use thereof. Also disclosed herein are diamino lipid (DAL) nanoparticles. These nanoparticles can encapsulate a nucleic acid, such as an mRNA, for example. This mRNA can encode a cytokine, such as IL-12, IL-27 and GM-CSF. Also disclosed are methods of treating a subject in need thereof, such as a subject with cancer.

### CROSS-REFERENCE TO RELATED APPLICATIONS

This application claims the benefit of priority to U.S. Provisional Application No. 63/286,272 filed Dec. 6, 2021, which is hereby incorporated herein by reference in its entirety.

### GOVERNMENT SUPPORT CLAUSE

This invention was made with government support under grant/contract numbers R01 CA229254 and R35 GM 119679 awarded by the National Institutes of Health. The government has certain rights in the invention.

### BACKGROUND

Improved methods and compositions for treatment of various diseases and disorders are continually needed. For example, cytokines are important immunotherapeutics with approved drugs for the treatment of human cancers. Despite efforts to develop systemic cytokine monotherapy for cancers, the systemic toxicity of cytokines and potent tumor local effects in induction of anti-tumor immunity have limited their use. Thus, the development of clinically relevant, cytokine-based local therapy is urgently needed.

The compounds, compositions, and methods disclosed herein address these and other needs.

### SUMMARY

In accordance with the purposes of the disclosed compositions and methods as embodied and broadly described herein, the disclosed subject matter relates to compounds and methods of making and use thereof.

For example, disclosed herein are compositions comprising a compound defined by Formula I, or a pharmaceutically acceptable salt thereof:

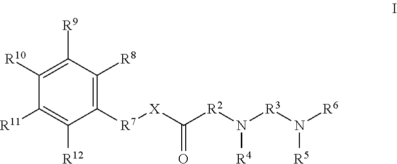

wherein
X is O, S, or NR¹;
R¹ is hydrogen, substituted or unsubstituted C₁-C₁₀ alkyl, or substituted or unsubstituted C₃-C₁₀ aryl group;
R² is substituted or unsubstituted C₁-C₁₀ alkyl, or substituted or unsubstituted C₁-C₁₀ alkenyl;
R³ is substituted or unsubstituted C₁-C₅ alkyl; and
R⁴, R⁵, and R⁶ are each independently substituted or unsubstituted C₆-C₂₀ alkyl;
R⁷ is substituted or unsubstituted C₁-C₅ alkyl;
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples, R² is a substituted or unsubstituted C₄-C₆ alkyl. In some examples, R² is a substituted or unsubstituted C₃ alkyl. In some examples, R² is an unsubstituted C₄-C₆ alkyl. In some examples, R² is an unsubstituted C₅ alkyl.

In some examples, R³ is a substituted or unsubstituted C₂-C₄ alkyl. In some examples, R³ is a substituted or unsubstituted C₃ alkyl. In some examples, R³ is an unsubstituted C₂-C₄ alkyl.

In some examples, R³ is an unsubstituted C₃ alkyl.

In some examples, R⁴, R⁵, and R⁶ are each independently a substituted or unsubstituted C₁₀-C₁₄ alkyl. In some examples, R⁴, R⁵, and R⁶ are each independently an unsubstituted C₁₀-C₁₄ alkyl. In some examples, R⁴, R⁵, and R⁶ are each independently a substituted or unsubstituted C₁₂ alkyl. In some examples, R⁴, R⁵, and R⁶ are the same. In some examples, R⁴, R⁵, and R⁶ are all an unsubstituted C₁₂ alkyl.

In some examples, the compound is defined by Formula II:

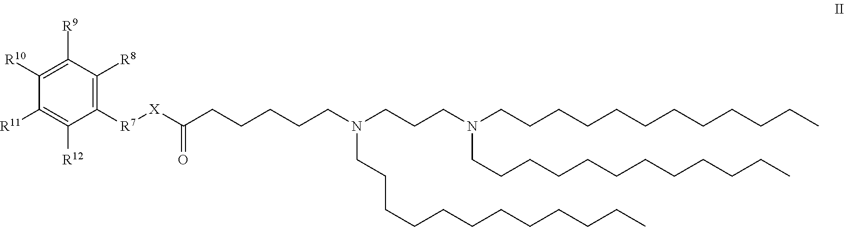

or a pharmaceutically acceptable salt thereof.

In some examples of Formula I or Formula II, X is NR¹.

In some examples, the compound is defined by Formula III:

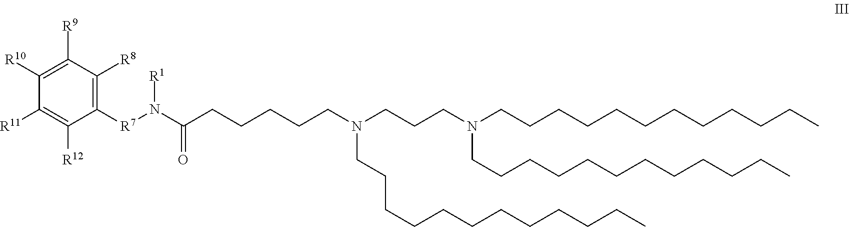

or a pharmaceutically acceptable salt thereof.

In some examples, the compound is defined by Formula IV:

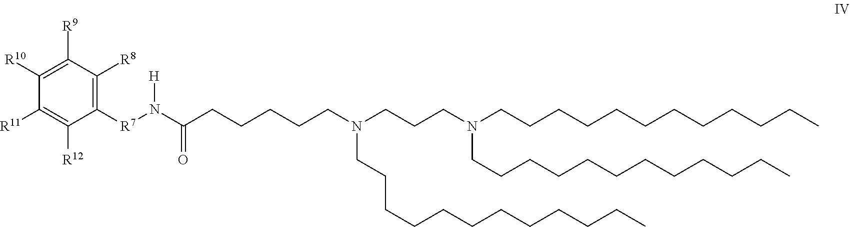

or a pharmaceutically acceptable salt thereof.

In some examples The composition of claim 17 or 18, wherein the compound is defined by Formula V:

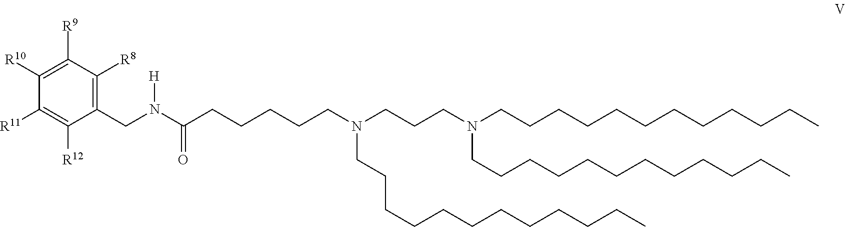

or a pharmaceutically acceptable salt thereof.

In some examples, the compound is defined by Formula VI:

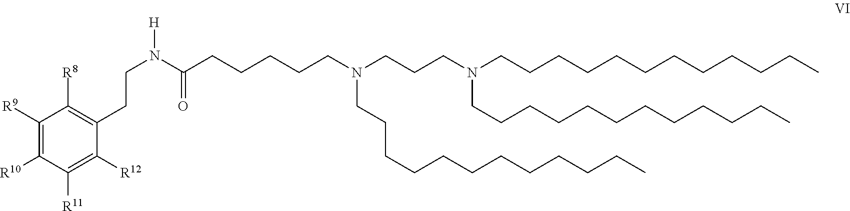

or a pharmaceutically acceptable salt thereof.

In some examples, the compound is defined by Formula VII, or a pharmaceutically acceptable salt thereof:

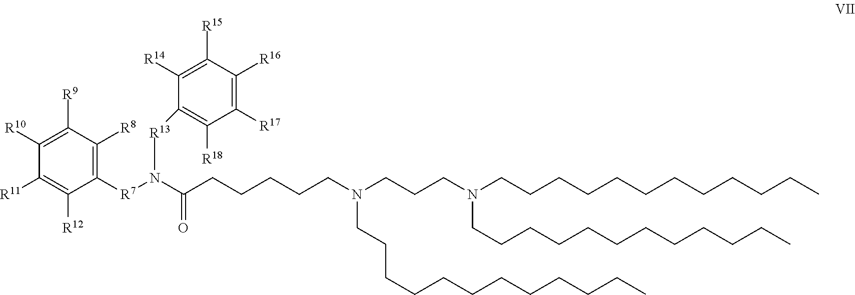

wherein
R¹³ is substituted or unsubstituted C₁-C₅ alkyl;
R¹⁴, R¹⁵, R¹⁶, R¹⁷, and R¹⁸ are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, substituted or unsubstituted C₁-C₂₀ acyl, or NR^{a}R^{b}, or wherein, as valence permits, R¹⁴ and R¹⁵, R¹⁵ and R¹⁶, R¹⁶ and R¹⁷, or R¹⁷ and R¹⁸, together with the atoms to which they are attached, form a 3-10 membered substituted or unsubstituted cyclic moiety optionally including from 1 to 3 heteroatoms; and
R^{a} and R^{b} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples, the compound is defined by Formula VIII:

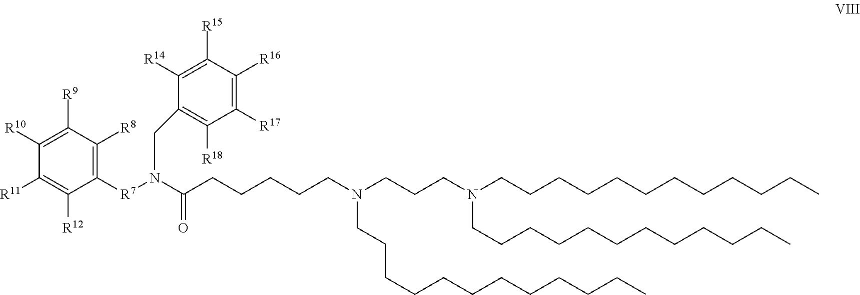

or a pharmaceutically acceptable salt thereof.

In some examples, the compound is selected from the group consisting of:

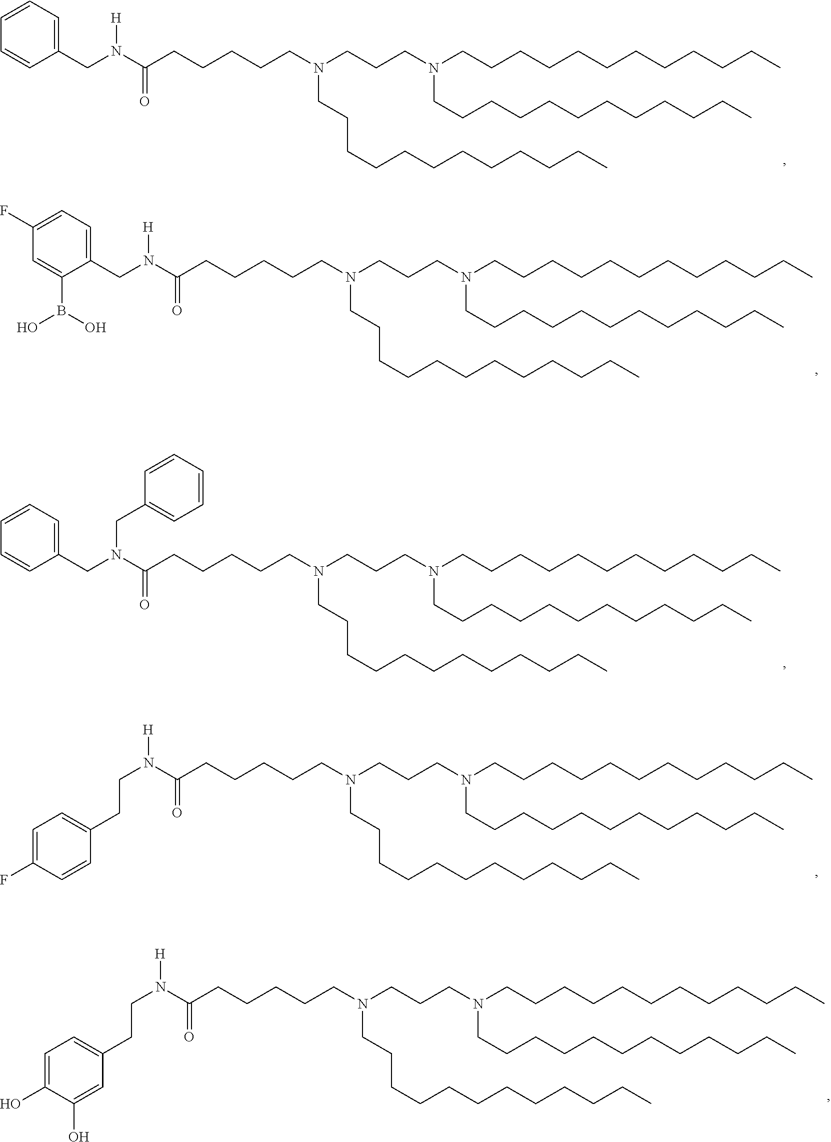 [SMILES: CCCCCCCCCCCCN(CCCCCCCCCCCC)CCCN(CCCCCCCCCCCC)CCCCCC(=O)NCc1ccccc1 ; CCCCCCCCCCCCN(CCCCCCCCCCCC)CCCN(CCCCCCCCCCCC)CCCCCC(=O)NCc1ccc(F)cc1B(O)O ; CCCCCCCCCCCCN(CCCCCCCCCCCC)CCCN(CCCCCCCCCCCC)CCCCCC(=O)N(Cc1ccccc1)Cc1ccccc1 ; CCCCCCCCCCCCN(CCCCCCCCCCCC)CCCN(CCCCCCCCCCCC)CCCCCC(=O)NCCc1ccc(F)cc1 ; CCCCCCCCCCCCN(CCCCCCCCCCCC)CCCN(CCCCCCCCCCCC)CCCCCC(=O)NCCc1ccc(O)c(O)c1]

or a pharmaceutically acceptable salt thereof.

In some examples, the compound comprises:

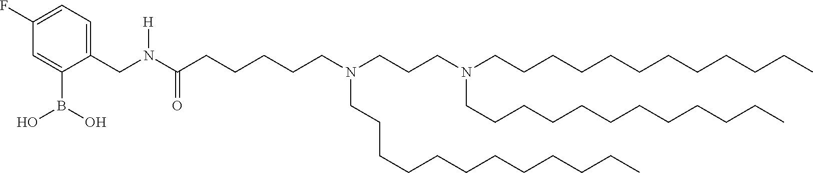 [SMILES: CCCCCCCCCCCCN(CCCCCCCCCCCC)CCCN(CCCCCCCCCCCC)CCCCCC(=O)NCc1ccc(F)cc1B(O)O]

or a pharmaceutically acceptable salt thereof.

Also disclosed herein are methods of making any of the compositions disclosed herein.

Also disclosed herein are lipid particles comprising any of the compositions disclosed herein. In some examples, the lipid particle is substantially spherical in shape. In some examples, the lipid particle has an average particle size of from 50 nanometers (nm) to 500 nm. In some examples, the lipid particle has an average particle size of from 100 nm to 200 nm, from 120 nm to 140 nm, or from 150 nm to 200 nm. In some examples, the lipid particle has a polydispersity index of 0.3 or less, 0.2 or less, or 0.1 or less. In some examples, the lipid particle further comprises an additional component. In some examples, the additional component comprises an additional lipid. In some examples, the additional lipid comprises a phospholipid, a sterol, or a combination thereof. In some examples, the lipid particle further comprises 1,2-dioleoyl-sn-glycero-3-phosphoethanolamine (DOPE), cholesterol, 1,2-dimyristoyl-rac-glycero-3-methylpolyoxyethylene, or a combination thereof.

Also disclosed herein are pharmaceutical compositions comprising a therapeutic agent encapsulated within any of the lipid particles disclosed herein. In some examples, the therapeutic agent is encapsulated within the lipid particle with an encapsulation efficiency of 50% or more, 75% or more, or 90% or more. In some examples, the therapeutic agent comprises an anticancer agent, an anti-inflammatory agent, an antimicrobial agent, or a combination thereof. In some examples, the therapeutic agent comprises a chemotherapeutic agent, an immunotherapeutic agent, or a combination thereof. In some examples, the therapeutic agent comprises a nucleic acid. In some examples, the nucleic acid is mRNA. In some examples, the mRNA encodes a cytokine.

Also disclosed herein are pharmaceutical compositions comprising mRNA encoding an immunotherapeutic agent encapsulated within a lipid particle. In some examples, the immunotherapeutic agent is a cytokine. In some examples, the lipid particle comprises any of the lipid particles disclosed herein. In some examples, the cytokine comprises IL-12, IL-27, GM-CSF, or a combination thereof.

Also disclosed herein are methods of making any of the pharmaceutical compositions disclosed herein.

Also disclosed herein are methods of treating a disease or disorder in a subject in need thereof, the methods comprising administering to the subject a therapeutically effective amount of any of the pharmaceutical compositions disclosed herein. In some examples, the disease comprises cancer.

Also disclosed herein are methods of suppressing tumor growth in a subject, comprising contacting at least a portion of the tumor with a therapeutically effective amount of any of the pharmaceutical compositions disclosed herein.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-FIG. 1B shows diamino lipid derivatives (DALs). (FIG. 1A) The structures of DALs. (FIG. 1B) Representative synthetic routes to DAL1 and DAL4.

FIG. 2A-FIG. 2G shows DAL-LNPs delivery of mRNA in vitro. (FIG. 2A) Encapsulation efficiency, (FIG. 2B) size distribution and polydispersity index (PDI) of DAL-LNPs encapsulating luciferase mRNA (DAL-LNP-Luc). (FIG. 2C) In vitro delivery of DAL-LNP-Luc in B16 cells. (FIG. 2D) Cryo-EM image of DAL4-LNP co-encapsulating IL 12 mRNA and IL27 mRNA. Bar=50 nm. In vitro delivery of DAL4-LNPs encapsulating either (FIG. 2E) IL-27 mRNA (DAL4-LNP-IL27), (FIG. 2F) IL-12 mRNA (DAL4-LNP-IL12), or (FIG. 2G) GM-CSF mRNA (DAL4-LNP-GM-CSF) in B16 cells. The proteins in the supernatants were determined by ELISA. All data are presented as the mean±S.D. Statistical significance in C was analyzed using one-way ANOVA. ***P<0.001.

FIG. 3A-FIG. 3D shows in vivo anti-cancer activity of DAL4-LNPs encapsulating single or multiple cytokine mRNAs. (FIG. 3A) Tumor size after treatment DAL4-LNPs loaded with single mRNA formulation (n=6). (FIG. 3B) Overall survival of tumor-bearing mice. (FIG. 3C) Tumor size after treatment with DAL4-LNPs formulation encapsulating two or three cytokine mRNAs (n=5-7). (FIG. 3D) Overall survival of tumor-bearing mice. Data in (FIG. 3A) and (FIG. 3C) are presented as the mean±S.E.M. Statistical significance in (FIG. 3A) and (FIG. 3C) was analyzed using two-way ANOVA with repeated measurements. Statistical significance in (FIG. 3B), (FIG. 3D) were analyzed using the log-rank (Mantel-Cox) test. *P <0.05; **P<0.01; ***P<0.001.

FIG. 4 shows a graphical view of diamino lipid derived nanoparticles.

FIG. 5A-FIG. 5B shows in vivo anti-cancer efficacy of DAL4-LNPs encapsulating single or multiple cytokine mRNAs. (FIG. 5A) Tumor size after treatment with DAL4-LNPs formulation encapsulating IL 12, IL27 or both IL 12+IL27 mRNAs (n=5-7). (FIG. 5B) Overall survival of tumor-bearing mice. Data in (FIG. 5A) are presented as the mean t S.E.M. Statistical significance in (FIG. 5A) was analyzed using two-way ANOVA with repeated measurements. Statistical significance in (FIG. 5B) were analyzed using the log-rank (Mantel-Cox) test. *P<0.05; **P<0.01; ***P<0.001; ns, not significant.

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of fibrotic conditions, “treating,” “treat,” and “treatment” as used herein, refers to partially or completely inhibiting or reducing the fibrotic condition which the subject is suffering. In one embodiment, this term refers to an action that occurs while a patient is suffering from, or is diagnosed with, the fibrotic condition, which reduces the severity of the condition, or retards or slows the progression of the condition. Treatment need not result in a complete cure of the condition; partial inhibition or reduction of the fibrotic condition is encompassed by this term.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “anticancer” refers to the ability to treat or control cellular proliferation and/or tumor growth at any concentration.

As used herein, “molecular weight” refers to number average molecular weight as measured by ¹H NMR spectroscopy, unless indicated otherwise.

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

As used herein, the term “encapsulation,” or grammatical equivalent, refers to the process of confining an individual nucleic acid molecule within a nanoparticle.

As used herein, “expression” of a mRNA refers to translation of an mRNA into a peptide (e.g., an antigen), polypeptide, or protein (e.g., an enzyme) and also can include, as indicated by context, the post-translational modification of the peptide, polypeptide or fully assembled protein (e.g., enzyme). In this application, the terms “expression” and “production,” and grammatical equivalent, are used inter-changeably.

As used herein, the term “messenger RNA (mRNA)” refers to a polynucleotide that encodes at least one peptide, polypeptide or protein. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Co-Cm preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acyl, alkyl, alkoxy, alkenyl, alkynyl, aryl, heteroaryl, aldehyde, amino, boronic acid, cyano, carboxylic acid, ester, ether, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, boronic acid, cyano, carboxylic acid, ester, ether, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acyl, aldehyde, amino, boronic acid, cyano, carboxylic acid, ester, ether, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acyl, aldehyde, amino, boronic acid, cyano, carboxylic acid, ester, ether, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, boronic acid, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, boronic acid, cyano, carboxylic acid, ester, ether, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

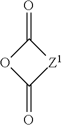

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “boronic acid” as used herein is represented by the formula —B(OH)₂.

The term “carboxylic acid” as used herein is represented by the formula —C(O)OH.

A “carboxylate” or “carboxyl” group as used herein is represented by the formula —C(O)O⁻.

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

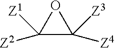

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Disclosed herein are compounds and methods of making and use thereof. For example, disclosed herein are compositions comprising a compound defined by Formula I, or a pharmaceutically acceptable salt thereof:

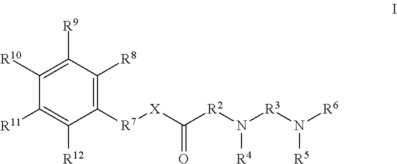

wherein
X is O, S, or NR¹;
R¹ is hydrogen, substituted or unsubstituted C₁-C₁₀ alkyl, or substituted or unsubstituted C₃-C₁₀ aryl group;
R² is substituted or unsubstituted C₁-C₁₀ alkyl, or substituted or unsubstituted C₁-C₁₀ alkenyl;
R³ is substituted or unsubstituted C₁-C₅ alkyl; and
R⁴, R⁵, and R⁶ are each independently substituted or unsubstituted C₆-C₂₀ alkyl; R⁷ is substituted or unsubstituted C₁-C₅ alkyl;
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula I, R² is a substituted or unsubstituted C₄-C₆ alkyl. In some examples of Formula I, R² is a substituted or unsubstituted C₅ alkyl. In some examples of Formula I, R² is an unsubstituted C₄-C₆ alkyl. In some examples of Formula I, R² is an unsubstituted C₅ alkyl.

In some examples of Formula I, R³ is a substituted or unsubstituted C₂-C₄ alkyl. In some examples of Formula I, R³ is a substituted or unsubstituted C₃ alkyl. In some examples of Formula I, R³ is an unsubstituted C₂-C₄ alkyl. In some examples of Formula I, R³ is an unsubstituted C₃ alkyl.

In some examples of Formula I, R⁴, R⁵, and R⁶ are each independently a substituted or unsubstituted C₁₀-C₁₄ alkyl. In some examples of Formula I, R⁴, R⁵, and R⁶ are each independently an unsubstituted C₁₀-C₁₄ alkyl. In some examples of Formula I, R⁴, R⁵, and R⁶ are each independently a substituted or unsubstituted C₁₂ alkyl. In some examples of Formula I, R⁴, R⁵, and R⁶ are the same. In some examples of Formula I, R⁴, R⁵, and R⁶ are all an unsubstituted C₁₂ alkyl.

In some examples of Formula I, R² is a substituted or unsubstituted C₄-C₆ alkyl and R³ is a substituted or unsubstituted C₂-C₄ alkyl. In some examples of Formula I, R² is an unsubstituted C₄-C₆ alkyl and R3 is an unsubstituted C₂-C₄ alkyl. In some examples of Formula I, R2 is an unsubstituted C₃ alkyl and R³ is an unsubstituted C₃ alkyl.

In some examples of Formula I, R² is a substituted or unsubstituted C₄-C₆ alkyl and R⁴, R⁵, and R⁶ are each independently a substituted or unsubstituted C₁₀-C₁₄ alkyl. In some examples of Formula I, R² is an unsubstituted C₄-C₆ alkyl and R⁴, R⁵, and R⁶ are each independently an unsubstituted C₁₀-C₁₄ alkyl. In some examples of Formula I, R² is an unsubstituted C₃ alkyl and R⁴, R⁵, and R⁶ are all an unsubstituted C₁₂ alkyl.

In some examples of Formula I, R³ is a substituted or unsubstituted C₂-C₄ alkyl and R⁴, R⁵, and R⁶ are each independently a substituted or unsubstituted C₁₀-C₁₄ alkyl. In some examples of Formula I, R³ is an unsubstituted C₂-C₄ alkyl and R⁴, R⁵, and R⁶ are each independently an unsubstituted C₁₀-C₁₄ alkyl. In some examples of Formula I, R³ is an unsubstituted C₃ alkyl and R⁴, R⁵, and R⁶ are all an unsubstituted C₁₂ alkyl.

In some examples of Formula I, R² is a substituted or unsubstituted C₄-C₆ alkyl; R³ is a substituted or unsubstituted C₂-C₄ alkyl; and R⁴, R⁵, and R⁶ are each independently a substituted or unsubstituted C₁₀-C₁₄ alkyl. In some examples of Formula I, R² is an unsubstituted C₄-C₆ alkyl; R³ is an unsubstituted C₂-C₄ alkyl; and R⁴, R⁵, and R⁶ are each independently an unsubstituted C₁₀-C₁₄ alkyl. In some examples of Formula I, R² is an unsubstituted C₅ alkyl; R³ is an unsubstituted C₃ alkyl; and R⁴, R⁵, and R⁶ are all an unsubstituted Cu alkyl.

In some examples of Formula I, X is O. In some examples of Formula I, X is NR¹. In some examples of Formula I, X is NR¹ and R¹ is hydrogen. In some examples of Formula I, X is NR¹ and R¹ is substituted or unsubstituted C₃-C₁₀ aryl group. In some examples of Formula I, X is NR¹ and R¹ and

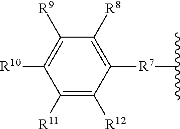

are the same.

In some examples of Formula I, R⁷ is substituted or unsubstituted C₁-C₃ alkyl. In some examples of Formula I, R⁷ is unsubstituted C₁-C₃ alkyl. In some examples of Formula I, R¹ is CH₂. In some examples of Formula I, R⁷ is CH₂CH₂.

In some examples of Formula I, R⁸, R⁹, R¹⁰, R¹¹, and R¹² are each independently H, OH, halogen, boronic acid, or substituted or unsubstituted C₁-C₂₀ alkyl. In some examples of Formula I, R⁸, R⁹, R¹⁰, R¹¹, and R¹² are each independently H, OH, halogen, boronic acid, or substituted or unsubstituted C₁-C₁₀ alkyl. In some examples of Formula I, R⁸, R⁹, R¹⁰, R¹¹, and R¹² are each independently H, OH, halogen, boronic acid, or substituted or unsubstituted C₁-C₅ alkyl. In some examples of Formula I, R⁸, R⁹, R¹⁰, R¹¹, and R¹² are each independently H, OH, halogen, or boronic acid.

In some examples of Formula I, R⁸ is hydrogen. In some examples of Formula I, R⁹ is hydrogen. In some examples of Formula I, R⁸ and R⁹ are both hydrogen.

In some examples of Formula I, R¹⁰ is halogen. In some examples of Formula I, R¹⁰ is F. In some examples of Formula I, R¹⁰ is OH. In some examples of Formula I, R¹⁰ is H.

In some examples of Formula I, R¹¹ is H. In some examples of Formula I, R¹¹ is OH.

In some examples of Formula I, R¹² is boronic acid. In some examples of Formula I, R² is H.

In some examples of Formula I, R⁸ and R⁹ are both hydrogen and R¹⁰ is halogen. In some examples of Formula I, R⁸ and R⁹ are both hydrogen and R¹⁰ is F.

In some examples of Formula I, R⁸, R⁹, and R¹¹ are all hydrogen. In some examples of Formula I, R⁸, R⁹, and R¹¹ are all hydrogen; R¹⁰ is halogen; and R² is boronic acid. In some examples of Formula I, R⁸, R⁹, and R¹¹ are all hydrogen; R¹⁰ is F; and R¹² is boronic acid.

In some examples of Formula I, R⁸, R⁹, R¹¹, and R¹² are all hydrogen. In some examples of Formula I, R⁸, R⁹, R¹¹, and R¹² are all hydrogen and R¹⁰ is halogen. In some examples of Formula I, R⁸, R⁹, R¹¹, and R¹² are all hydrogen and R¹² is F.

In some examples of Formula I, R⁸, R⁹, and R¹² are all hydrogen. In some examples of Formula I, R¹⁰ and R¹¹ are both OH. In some examples of Formula I, R⁸, R⁹, and R¹² are all hydrogen and R¹⁰ and R¹¹ are both OH.

In some examples of Formula I, R⁸, R⁹, R¹⁰, R¹¹, and R¹² are all hydrogen.

In some examples, the compound is defined by Formula II or a pharmaceutically acceptable salt thereof:

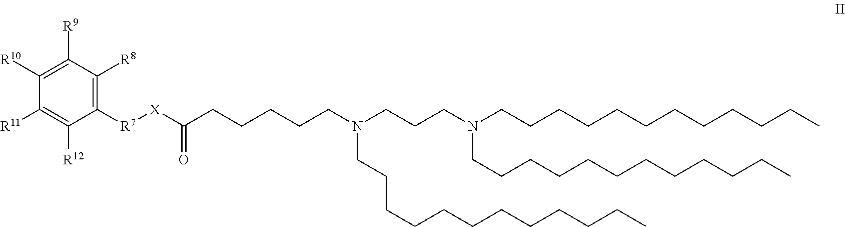

wherein
X is O, S, or NR¹;
R¹ is hydrogen, substituted or unsubstituted C₁-C₁₀ alkyl, or substituted or unsubstituted C₃-C₁₀ aryl group;
R⁷ is substituted or unsubstituted C₁-C₅ alkyl;
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula II, X is O. In some examples of Formula II, X is NR¹. In some examples of Formula II, X is NR¹ and R¹ is hydrogen. In some examples of Formula H, X is NR¹ and R¹ is substituted or unsubstituted C₃-C₁₀ aryl group. In some examples of Formula H, X is NR¹ and R¹ and

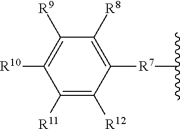

are the same.

In some examples of Formula II, R⁷ is substituted or unsubstituted C₁-C₃ alkyl. In some examples of Formula II, R⁷ is unsubstituted C₁-C₃ alkyl. In some examples of Formula II, R⁷ is CH₂. In some examples of Formula II, R⁷ is CH₂CH₂.

In some examples of Formula II, R⁸, R⁹, R¹⁰, R¹¹, and R¹² are each independently H, OH, halogen, boronic acid, or substituted or unsubstituted C₁-C₂₀ alkyl. In some examples of Formula H, R⁸, R⁹, R¹⁰, R¹¹, and R¹² are each independently H, OH, halogen, boronic acid, or substituted or unsubstituted C₁-C₁₀ alkyl. In some examples of Formula II, R⁸, R⁹, R¹⁰, R¹¹, and R¹² are each independently H, OH, halogen, boronic acid, or substituted or unsubstituted C₁-C₅ alkyl. In some examples of Formula II, R⁸, R⁹, R¹⁰, R¹¹, and R¹² are each independently H, OH, halogen, or boronic acid.

In some examples of Formula II, R⁸ is hydrogen. In some examples of Formula II, R⁹ is hydrogen. In some examples of Formula H, R⁸ and R⁹ are both hydrogen.

In some examples of Formula II, R¹⁰ is halogen. In some examples of Formula II, R¹⁰ is F. In some examples of Formula II, R¹⁰ is OH. In some examples of Formula II, R¹¹ is H.

In some examples of Formula II, R¹¹ is H. In some examples of Formula II, R¹¹ is OH.

In some examples of Formula II, R¹² is boronic acid. In some examples of Formula II, R¹² is H.

In some examples of Formula II, R⁸ and R⁹ are both hydrogen and R¹⁰ is halogen. In some examples of Formula II, R⁸ and R⁹ are both hydrogen and R¹⁰ is F.

In some examples of Formula II, R⁸, R⁹, and R¹¹ are all hydrogen. In some examples of Formula H, R⁸, R⁹, and R¹¹ are all hydrogen; R¹⁰ is halogen; and R² is boronic acid. In some examples of Formula II, R⁸, R⁹, and R¹¹ are all hydrogen; R¹⁰ is F; and R¹² is boronic acid.

In some examples of Formula II, R⁸, R⁹, R¹, and R² are all hydrogen. In some examples of Formula II, R⁸, R⁹, R¹¹, and R¹² are all hydrogen and R¹⁰ is halogen. In some examples of Formula II, R⁸, R⁹, R¹¹, and R¹² are all hydrogen and R¹⁰ is F.

In some examples of Formula II, R⁸, R⁹, and R¹² are all hydrogen. In some examples of Formula II, R¹⁰ and R¹¹ are both OH. In some examples of Formula II, R⁸, R⁹, and R¹² are all hydrogen and R¹⁰ and R¹¹ are both OH.

In some examples of Formula II, R⁸, R⁹, R¹⁰, R¹¹, and R¹² are all hydrogen.

In some examples, the compound is defined by Formula III, or a pharmaceutically acceptable salt thereof:

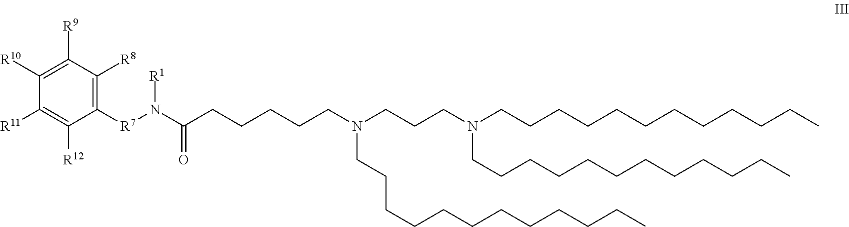

wherein
R¹ is hydrogen, substituted or unsubstituted C₁-C₁₀ alkyl, or substituted or unsubstituted C₃-C₁₀ aryl group;
R⁷ is substituted or unsubstituted C₁-C₅ alkyl;
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula III, R¹ is hydrogen. In some examples of Formula III, R¹ is substituted or unsubstituted C₃-C₁₀ aryl group. In some examples of Formula III, R¹ and

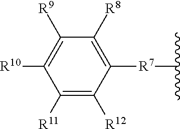

are the same.

In some examples of Formula III, R⁷ is substituted or unsubstituted C₁-C₃ alkyl. In some examples of Formula III, R⁷ is unsubstituted C₁-C₃ alkyl. In some examples of Formula III, R⁷ is CH₂. In some examples of Formula III, R⁷ is CH₂CH₂.

In some examples of Formula III, R⁸, R⁹, R¹⁰, R¹¹, and R¹² are each independently H, OH, halogen, boronic acid, or substituted or unsubstituted C₁-C₂₀ alkyl. In some examples of Formula III, R⁸, R⁹, R¹⁰, R¹¹, and R¹² are each independently H, OH, halogen, boronic acid, or substituted or unsubstituted C₁-C₁₀ alkyl. In some examples of Formula II, R, R⁹, R¹⁰, R¹⁰, and R¹² are each independently H, OH, halogen, boronic acid, or substituted or unsubstituted C₁-C₅ alkyl. In some examples of Formula II, R⁸, R⁹, R¹⁰, R¹¹, and R¹² are each independently H, OH, halogen, or boronic acid.

In some examples of Formula III, R⁸ is hydrogen. In some examples of Formula III, R⁹ is hydrogen. In some examples of Formula III, R⁸ and R⁹ are both hydrogen.

In some examples of Formula III, R¹⁰ is halogen. In some examples of Formula III, R¹⁰ is F. In some examples of Formula III, R¹⁰ is OH. In some examples of Formula III, R¹⁰ is H.

In some examples of Formula III, R¹¹ is H. In some examples of Formula III, R¹¹ is OH.

In some examples of Formula III, R¹² is boronic acid. In some examples of Formula III, R¹² is H.

In some examples of Formula III, R⁸ and R⁹ are both hydrogen and R¹⁰ is halogen. In some examples of Formula III, R⁸ and R⁹ are both hydrogen and Rio is F.

In some examples of Formula III, R⁸, R⁹, and R¹¹ are all hydrogen. In some examples of Formula III, R¹, R⁹, and R¹¹ are all hydrogen; R¹⁰ is halogen; and R¹² is boronic acid. In some examples of Formula III, R⁸, R¹⁰, and R¹¹ are all hydrogen; R¹⁰ is F; and R¹² is boronic acid.

In some examples of Formula III, R⁸, R⁹, R¹¹, and R¹² are all hydrogen. In some examples of Formula III, R⁸, R⁹, R¹¹, and R¹² are all hydrogen and R¹⁰ is halogen. In some examples of Formula III, R⁸, R⁹, R¹¹, and R¹² are all hydrogen and R¹⁰ is F.

In some examples of Formula III, R⁸, R⁹, and R¹² are all hydrogen. In some examples of Formula III, R¹⁰ and R¹¹ are both OH. In some examples of Formula III, R⁸, R⁹, and R² are all hydrogen and R¹⁰ and R¹¹ are both OH.

In some examples of Formula III, R⁸, R⁹, R¹⁰, R¹¹, and R¹² are all hydrogen.

In some examples, the compound is defined by Formula IV, or a pharmaceutically acceptable salt thereof:

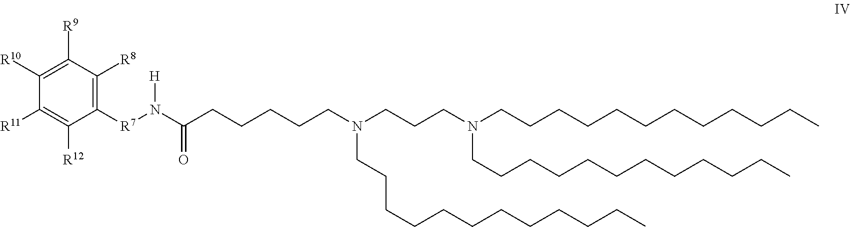

wherein
R⁷ is substituted or unsubstituted C₁-C₃ alkyl;
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{Y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula IV, R⁷ is substituted or unsubstituted C₁-C₃ alkyl. In some examples of Formula IV, R⁷ is unsubstituted C₁-C₃ alkyl. In some examples of Formula IV, R⁷ is CH₂. In some examples of Formula IV, R⁷ is CH₂CH₂.

In some examples of Formula IV, R⁸, R⁹, R¹⁰, R¹¹, and R¹² are each independently H, OH, halogen, boronic acid, or substituted or unsubstituted C₁-C₂₀ alkyl. In some examples of Formula IV, R⁸, R⁹, R¹⁰, R¹¹, and R¹² are each independently H, OH, halogen, boronic acid, or substituted or unsubstituted C₁-C₁₀ alkyl. In some examples of Formula IV, R⁸, R⁹, R¹⁰, R¹¹, and R¹² are each independently H, OH, halogen, boronic acid, or substituted or unsubstituted C₁-C₅ alkyl. In some examples of Formula IV, R⁸, R⁹, R¹⁰, R¹¹, and R¹² are each independently H, OH, halogen, or boronic acid.

In some examples of Formula IV, R⁸ is hydrogen. In some examples of Formula IV, R⁹ is hydrogen. In some examples of Formula IV, R⁸ and R⁹ are both hydrogen.

In some examples of Formula IV, R¹⁰ is halogen. In some examples of Formula IV, R¹⁰ is F. In some examples of Formula IV, R¹⁰ is OH. In some examples of Formula IV, R¹⁰ is H.

In some examples of Formula IV, R¹¹ is H. In some examples of Formula IV, R¹¹ is OH.

In some examples of Formula IV, R¹² is boronic acid. In some examples of Formula IV, R¹² is H.

In some examples of Formula IV, R⁸ and R⁹ are both hydrogen and R¹⁰ is halogen. In some examples of Formula IV, R⁸ and R^{v} are both hydrogen and R¹⁰ is F.

In some examples of Formula IV, R⁸, R⁹, and R¹¹ are all hydrogen. In some examples of Formula IV, R⁸, R⁹, and R¹¹ are all hydrogen; R¹⁰ is halogen; and R¹² is boronic acid. In some examples of Formula IV, R⁸, R⁹, and R¹¹ are all hydrogen; R¹⁰ is F; and R¹² is boronic acid.

In some examples of Formula IV, R⁸, R⁹, R¹¹, and R¹² are all hydrogen. In some examples of Formula IV, R⁸, R⁹, R¹¹, and R¹² are all hydrogen and R¹⁰ is halogen. In some examples of Formula IV, R⁸, R⁹, R¹¹, and R¹² are all hydrogen and R¹⁰ is F.

In some examples of Formula IV, R⁸, R⁹, and R¹² are all hydrogen. In some examples of Formula IV, R¹⁰ and R¹¹ are both OH. In some examples of Formula IV, R⁸, R⁹, and R² are all hydrogen and R¹⁰ and R¹¹ are both OH.

In some examples of Formula IV, R⁸, R⁹, R¹⁰, R¹¹, and R¹² are all hydrogen.

In some examples, the compound is defined by Formula V, or a pharmaceutically acceptable salt thereof:

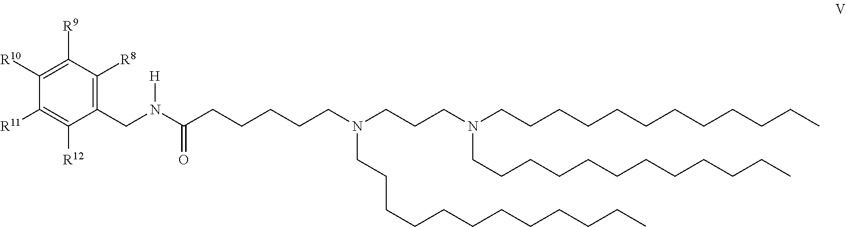

wherein
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula V, R⁸, R⁹, R¹⁰, R¹¹, and R¹² are each independently H, OH, halogen, boronic acid, or substituted or unsubstituted C₁-C₂₀ alkyl. In some examples of Formula V, R⁸, R⁹, R¹⁰, R¹¹, and R¹² are each independently H, OH, halogen, boronic acid, or substituted or unsubstituted C₁-C₁₀ alkyl. In some examples of Formula V, R⁸, R⁹, R¹⁰, R¹¹, and R¹² are each independently H, OH, halogen, boronic acid, or substituted or unsubstituted C₁-C₅ alkyl. In some examples of Formula V, R⁸, R⁹, R¹⁰, R¹¹, and R¹² are each independently H, OH, halogen, or boronic acid.

In some examples of Formula V, R⁸ is hydrogen. In some examples of Formula V, R⁹ is hydrogen. In some examples of Formula V, R⁸ and R⁹ are both hydrogen.

In some examples of Formula V, R¹⁰ is halogen. In some examples of Formula V, R¹⁰ is F. In some examples of Formula V, R¹⁰ is OH. In some examples of Formula V, R¹⁰ is H.

In some examples of Formula V, R¹¹ is H. In some examples of Formula V, R¹¹ is OH.

In some examples of Formula V, R¹² is boronic acid. In some examples of Formula V, R¹² is H.

In some examples of Formula V, R⁸ and R⁹ are both hydrogen and R¹⁰ is halogen. In some examples of Formula V, R⁸ and R⁹ are both hydrogen and R¹⁰ is F.

In some examples of Formula V, R⁸, R⁹, and R¹¹ are all hydrogen. In some examples of Formula V, R⁸, R⁹, and R¹¹ are all hydrogen; R¹⁰ is halogen; and R¹² is boronic acid. In some examples of Formula V, R⁸, R⁹, and R¹¹ are all hydrogen; R¹⁰ is F; and R¹² is boronic acid.

In some examples of Formula V, R⁸, R⁹, R¹¹, and R¹² are all hydrogen. In some examples of Formula V, R⁸, R⁹, R¹, and R² are all hydrogen and R¹⁰ is halogen. In some examples of Formula V, R⁸, R⁹, R¹¹, and R¹² are all hydrogen and R¹⁰ is F.

In some examples of Formula V, R^{a}, R⁹, and R¹² are all hydrogen. In some examples of Formula V, R¹⁰ and R¹¹ are both OH. In some examples of Formula V, R⁸, R⁹, and R¹² are all hydrogen and R¹⁰ and R¹¹ are both OH.

In some examples of Formula V, R⁸, R⁹, R¹⁰, R¹¹, and R¹² are all hydrogen.

In some examples, the compound is defined by Formula VI, or a pharmaceutically acceptable salt thereof:

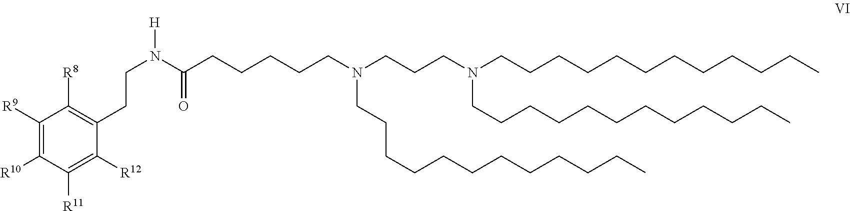

wherein
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula VI, R⁸, R⁹, R¹⁰, R¹¹, and R¹² are each independently H, OH, halogen, boronic acid, or substituted or unsubstituted C₁-C₂₀ alkyl. In some examples of Formula VI, R⁸, R⁹, R¹⁰, R¹¹, and R¹² are each independently H, OH, halogen, boronic acid, or substituted or unsubstituted C₁-C₁₀ alkyl. In some examples of Formula VI, R⁸, R⁹, R¹⁰, R¹¹, and R¹² are each independently H, OH, halogen, boronic acid, or substituted or unsubstituted C₁-C₅ alkyl. In some examples of Formula VI, R⁸, R⁹, R¹⁰, R¹¹, and R¹² are each independently H, OH, halogen, or boronic acid.

In some examples of Formula VI, R⁸ is hydrogen. In some examples of Formula VI, R⁹ is hydrogen. In some examples of Formula VI, R⁸ and R⁹ are both hydrogen.

In some examples of Formula VI, R¹⁰ is halogen. In some examples of Formula VI, R¹⁰ is F. In some examples of Formula VI, R¹⁰ is OH. In some examples of Formula VI, R¹⁰ is H.

In some examples of Formula VI, R¹¹ is H. In some examples of Formula VI, R¹¹ is OH.

In some examples of Formula VI, R¹² is boronic acid. In some examples of Formula VI, R¹² is H.

In some examples of Formula VI, R⁸ and R⁹ are both hydrogen and R¹⁰ is halogen. In some examples of Formula VI, R⁸ and R⁹ are both hydrogen and R¹⁰ is F.

In some examples of Formula VI, R⁸, R⁹, and R¹¹ are all hydrogen. In some examples of Formula VI, R⁸, R⁹, and R¹¹ are all hydrogen; R¹⁰ is halogen; and R² is boronic acid. In some examples of Formula VI, R⁸, R⁹, and R¹¹ are all hydrogen; R¹⁰ is F; and R¹² is boronic acid.

In some examples of Formula VI, R⁸, R⁹, R¹¹, and R¹² are all hydrogen. In some examples of Formula VI, R⁸, R⁹, R¹¹, and R¹² are all hydrogen and R¹⁰ is halogen. In some examples of Formula VI, R⁸, R⁹, R¹¹, and R¹² are all hydrogen and R¹⁰ is F.

In some examples of Formula VI, R, R⁹, and R¹² are all hydrogen. In some examples of Formula VI, R¹⁰ and R¹¹ are both OH. In some examples of Formula VI, R⁸, R⁹, and R² are all hydrogen and R¹⁰ and R¹¹ are both OH.

In some examples of Formula VI, R⁸, R⁹, R¹⁰, R¹¹, and R¹² are all hydrogen.

In some examples, the compound is defined by Formula VII, or a pharmaceutically acceptable salt thereof:

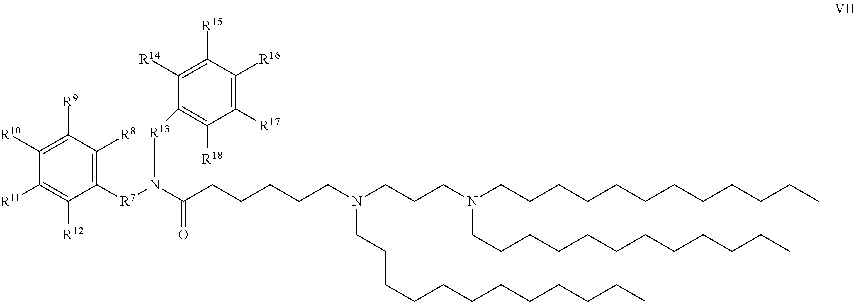

wherein
R⁶ is substituted or unsubstituted C₁-C₅ alkyl;
R⁸, R⁹, R¹⁰, R¹¹, and R² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰, and R¹¹, or R¹¹ and R¹², together with the atoms to which they are attached, form a 3-10 membered substituted or unsubstituted cyclic moiety optionally including from 1 to 3 heteroatoms; R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl;
R¹³ is substituted or unsubstituted C₁-C₅ alkyl;
R¹⁴, R¹⁵, R¹⁶, R¹⁷, and R¹⁸ are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, substituted or unsubstituted C₁-C₂₀ acyl, or NR^{a}R^{b}, or wherein, as valence permits, R¹⁴ and R¹⁵, R¹⁵ and R¹⁶, R¹⁶ and R¹⁷, or R¹⁷ and R¹⁸, together with the atoms to which they are attached, form a 3-10 membered substituted or unsubstituted cyclic moiety optionally including from 1 to 3 heteroatoms; and
R^{a} and R^{b} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula VII, R⁷ is substituted or unsubstituted C₁-C₃ alkyl. In some examples of Formula VII, R⁷ is unsubstituted C₁-C₃ alkyl. In some examples of Formula VII, R⁷ is CH₂. In some examples of Formula VII, R⁷ is CH₂CH₂.

In some examples of Formula VII, R¹³ is substituted or unsubstituted C₁-C₃ alkyl. In some examples of Formula VII, R¹³ is unsubstituted C₁-C₃ alkyl. In some examples of Formula VII, R¹³ is CH₂. In some examples of Formula VII, R¹³ is CH₂CH₂.

In some examples of Formula VII, R⁷ and R¹³ are different. In some examples of Formula VII, R⁷ and R¹³ are the same.

In some examples of Formula VII, R⁷ is substituted or unsubstituted C₁-C₃ alkyl and R¹³ is substituted or unsubstituted C₁-C₃ alkyl. In some examples of Formula VII, R⁶ is unsubstituted C₁-C₃ alkyl and R³ is unsubstituted C₁-C₃ alkyl. In some examples of Formula VII, R⁷ is CH₂ and R¹³ is CH₂.

In some examples of Formula VII,

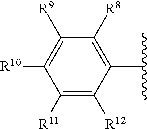

and

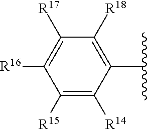

are different. In some examples of Formula VII,

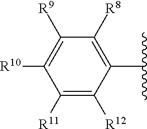

and

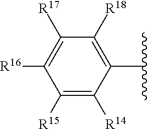

are the same.

In some examples of Formula VII,

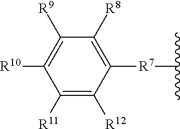

and

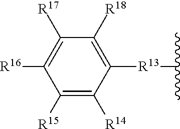

are different.

In some examples of Formula VII,

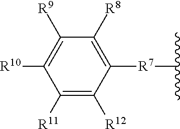

and

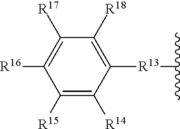

are the same.

In some examples of Formula VII, R⁸, R⁹, R¹⁰, R¹¹, and R¹² are each independently H, OH, halogen, boronic acid, or substituted or unsubstituted C₁-C₂₀ alkyl. In some examples of Formula VII, R⁸, R⁹, R¹⁰, R¹¹, and R¹² are each independently H, OH, halogen, boronic acid, or substituted or unsubstituted C₁-C₁₀ alkyl. In some examples of Formula VII, R⁸, R⁹, R¹⁰, R¹¹, and R¹² are each independently H, OH, halogen, boronic acid, or substituted or unsubstituted C₁-C₅ alkyl. In some examples of Formula VII, R⁸, R⁹, R¹⁰, R¹¹, and R¹² are each independently H, OH, halogen, or boronic acid.

In some examples of Formula VII, R⁸ is hydrogen. In some examples of Formula VII, R⁹ is hydrogen. In some examples of Formula VII, R³ and R⁹ are both hydrogen.

In some examples of Formula VII, R¹⁰ is halogen. In some examples of Formula VII, R¹⁰ is F. In some examples of Formula VII, R¹⁰ is OH. In some examples of Formula VII, R¹⁰ is H.

In some examples of Formula VII, R¹¹ is H. In some examples of Formula VII, R¹¹ is OH.

In some examples of Formula VII, R¹² is boronic acid. In some examples of Formula VII, R¹² is H.

In some examples of Formula VII, R⁸ and R⁹ are both hydrogen and R¹⁰ is halogen. In some examples of Formula VII, R⁸ and R⁹ are both hydrogen and R¹⁰ is F.

In some examples of Formula VII, R⁸, R⁹, and R¹¹ are all hydrogen. In some examples of Formula VII, R⁸, R⁹, and R¹¹ are all hydrogen; R¹⁰ is halogen; and R¹² is boronic acid. In some examples of Formula VII, R⁸, R⁹, and R¹¹ are all hydrogen; R¹⁰ is F; and R¹² is boronic acid.

In some examples of Formula VII, R⁸, R⁹, R¹¹, and R¹² are all hydrogen. In some examples of Formula VII, R³, R⁹, R¹¹, and R¹² are all hydrogen and R¹⁰ is halogen. In some examples of Formula VII, R⁸, R⁹, R¹¹, and R¹² are all hydrogen and R¹⁰ is F.

In some examples of Formula VII, R⁸, R⁹, and R¹² are all hydrogen. In some examples of Formula VII, R¹⁰ and R¹¹ are both OH. In some examples of Formula VII, R⁸, R⁹, and R¹² are all hydrogen and R¹⁰ and R¹¹ are both OH.

In some examples of Formula VII, R⁸, R⁹, R¹⁰, R¹¹, and R¹² are all hydrogen.

In some examples of Formula VII, R¹⁸, R¹⁷, R¹⁶, R¹⁵, and R¹⁴ are each independently H, OH, halogen, boronic acid, or substituted or unsubstituted C₁-C₂₀ alkyl. In some examples of Formula VII, R¹⁸, R¹⁷, R¹⁶, R¹⁵, and R¹⁴ are each independently H, OH, halogen, boronic acid, or substituted or unsubstituted C₁-C₁₀ alkyl. In some examples of Formula VII, R¹⁸, R¹⁷, R¹⁶, R¹⁵, and R¹⁴ are each independently H, OH, halogen, boronic acid, or substituted or unsubstituted C₁-C₅ alkyl. In some examples of Formula VII, R¹⁸, R¹⁷, R¹⁶, R¹⁵, and R¹⁴ are each independently H, OH, halogen, or boronic acid.

In some examples of Formula VII, R¹⁸ is hydrogen. In some examples of Formula VII, R¹⁷ is hydrogen. In some examples of Formula VII, R¹⁸ and R¹⁷ are both hydrogen.

In some examples of Formula VII, R¹⁶ is halogen. In some examples of Formula VII, R¹⁶ is F. In some examples of Formula VII, R¹⁶ is OH. In some examples of Formula VII, R¹⁶ is H.

In some examples of Formula VII, R¹⁵ is H. In some examples of Formula VII, R¹⁵ is OH.

In some examples of Formula VII, R¹⁴ is boronic acid. In some examples of Formula VII, R¹⁴ is H.

In some examples of Formula VII, R¹⁸ and R¹⁷ are both hydrogen and R¹⁶ is halogen. In some examples of Formula VII, R¹⁸ and R¹⁷ are both hydrogen and R¹⁶ is F.

In some examples of Formula VII, R^{18S}, R¹⁷, and R¹⁵ are all hydrogen. In some examples of Formula VII, R¹⁸, R¹⁷, and R¹⁵ are all hydrogen; R¹⁶ is halogen; and R¹⁴ is boronic acid. In some examples of Formula VII, R¹⁸, R¹⁷, and R¹⁵ are all hydrogen; R¹⁶ is F; and R¹⁴ is boronic acid.

In some examples of Formula VII, R¹⁸, R¹⁷, R¹⁵, and R¹⁴ are all hydrogen. In some examples of Formula VII, R¹⁸, R¹⁷, R¹⁵, and R¹⁴ are all hydrogen and R¹⁶ is halogen. In some examples of Formula VII, R¹⁸, R¹⁷, R¹⁵, and R¹⁴ are all hydrogen and R¹⁶ is F.

In some examples of Formula VII, R¹⁸, R¹⁷, and R¹⁴ are all hydrogen. In some examples of Formula VII, R¹⁶ and R¹⁵ are both OH. In some examples of Formula VII, R¹⁸, R¹⁷, and R¹⁴ are all hydrogen and R¹⁶ and R¹⁵ are both OH.

In some examples of Formula VII, R¹⁸, R¹⁷, R¹⁶, R¹⁵, and R¹⁴ are all hydrogen.

In some examples of Formula VII, R⁸, R⁹, R¹⁰, R¹¹, R¹², R¹⁴, R¹⁵, R¹⁶, R¹⁷, and R¹⁸ are each independently H, OH, halogen, boronic acid, or substituted or unsubstituted C₁-C₂₀ alkyl. In some examples of Formula VII, R⁸, R⁹, R¹⁰, R¹¹, R¹², R¹⁴, R¹⁵, R¹⁶, R¹⁷, and R¹⁸ are each independently H, OH, halogen, boronic acid, or substituted or unsubstituted C₁-C₁₀ alkyl. In some examples of Formula VII, R⁸, R⁹, R¹⁰, R¹¹, R¹², R¹⁴, R¹⁵, R¹⁶, R¹⁷, and R¹⁸ are each independently H, OH, halogen, boronic acid, or substituted or unsubstituted C₁-C₅ alkyl. In some examples of Formula VII, R⁸, R⁹, R¹⁰, R¹¹, R¹², R¹⁴, R¹⁵, R¹⁶, R¹⁷, and R¹⁸ are each independently H, OH, halogen, or boronic acid. In some examples of Formula VII, R⁸, R⁹, R¹⁰, R¹¹, R¹², R¹⁴, R¹⁵, R¹⁶, R¹⁷, and R¹⁸ are all hydrogen.

In some examples, the compound is defined by Formula VIII, or a pharmaceutically acceptable salt thereof:

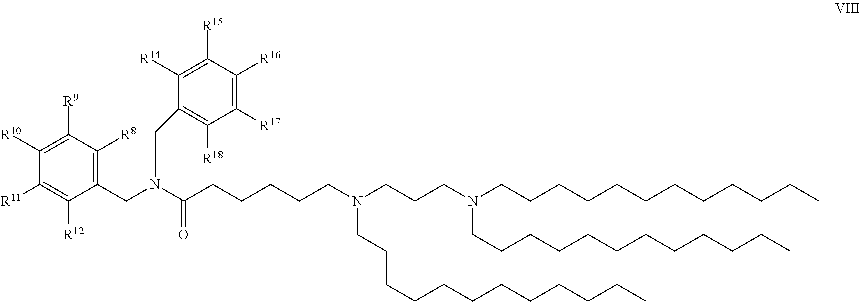

wherein
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR^{x}R^{y}, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms;
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl;
R¹⁴, R¹⁵, R¹⁶, R¹⁷, and R¹⁸ are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, substituted or unsubstituted C₁-C₂₀ acyl, or NR^{a}R^{b}, or wherein, as valence permits, R¹⁴ and R¹⁵, R¹⁵ and R¹⁵, R¹⁶ and R¹⁷, or R¹⁷ and R¹⁸, together with the atoms to which they are attached, form a 3-10 membered substituted or unsubstituted cyclic moiety optionally including from 1 to 3 heteroatoms; and
R^{a} and R^{b} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

In some examples of Formula VIII,

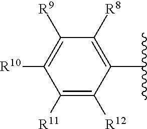

and

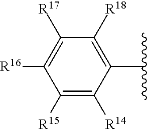

are different. In some examples of Formula VIII,

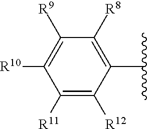

and

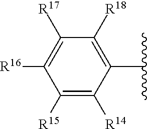

are the same.

In some examples of Formula VIII, R⁸, R⁹, R¹⁰, R¹¹, and R¹² are each independently H, OH, halogen, boronic acid, or substituted or unsubstituted C₁-C₂₀ alkyl. In some examples of Formula VIII, R⁸, R⁹, R¹⁰, R¹¹, and R¹² are each independently H, OH, halogen, boronic acid, or substituted or unsubstituted C₁-C₁₀ alkyl. In some examples of Formula VIII, R⁸, R⁹, R¹⁰, R¹¹, and R² are each independently H, OH, halogen, boronic acid, or substituted or unsubstituted C₁-C₅ alkyl. In some examples of Formula VIII, R⁸, R⁹, R¹⁰, R¹¹, and R¹² are each independently H, OH, halogen, or boronic acid.

In some examples of Formula VIII, R⁸ is hydrogen. In some examples of Formula VIII, R⁹ is hydrogen. In some examples of Formula VIII, R^{a} and R⁹ are both hydrogen.

In some examples of Formula VIII, R¹⁰ is halogen. In some examples of Formula VIII, R¹⁰ is F. In some examples of Formula VIII, R¹⁰ is OH. In some examples of Formula VIII, R¹⁰ is H.

In some examples of Formula VIII, R¹¹ is H. In some examples of Formula VIII, R¹¹ is OH.

In some examples of Formula VIII, R¹² is boronic acid. In some examples of Formula VIII, R¹² is H.

In some examples of Formula VIII, R⁸ and R⁹ are both hydrogen and R¹⁰ is halogen. In some examples of Formula VIII, R⁸ and R⁹ are both hydrogen and R¹⁰ is F.

In some examples of Formula VIII, R⁸, R⁹, and R¹¹ are all hydrogen. In some examples of Formula VIII, R⁸, R⁹, and R¹¹ are all hydrogen; R¹⁰ is halogen; and R¹² is boronic acid. In some examples of Formula VIII, R⁸, R⁹, and R¹¹ are all hydrogen; R¹⁰ is F; and R¹² is boronic acid.

In some examples of Formula VIII, R⁸, R⁹, R¹¹, and R¹² are all hydrogen. In some examples of Formula VIII, R⁸, R⁹, R¹¹, and R¹² are all hydrogen and R¹⁰ is halogen. In some examples of Formula VIII, R⁸, R⁹, R¹¹, and R¹² are all hydrogen and R¹⁰ is F.

In some examples of Formula VIII, R⁸, R⁹, and R¹² are all hydrogen. In some examples of Formula VIII, R¹⁰ and R¹¹ are both OH. In some examples of Formula VIII, R⁸, R⁹, and R¹² are all hydrogen and R¹⁰ and R¹¹ are both OH.

In some examples of Formula VIII, R⁸, R⁹, R¹⁰, R¹¹, and R¹² are all hydrogen.

In some examples of Formula VIII, R¹⁸, R¹⁷, R¹⁶, R¹⁵, and R¹⁴ are each independently H, OH, halogen, boronic acid, or substituted or unsubstituted C₁-C₂₀ alkyl. In some examples of Formula VIII, R¹⁸, R¹⁷, R¹⁶, R¹⁵, and R¹⁴ are each independently H, OH, halogen, boronic acid, or substituted or unsubstituted C₁-C₁₀ alkyl. In some examples of Formula VIII, R¹⁸, R¹⁷, R¹⁶, R¹⁵, and R¹⁴ are each independently H, OH, halogen, boronic acid, or substituted or unsubstituted C₁-C₅ alkyl. In some examples of Formula VIII, R¹⁸, R¹⁷, R¹⁶, R¹⁵, and R¹⁴ are each independently H, OH, halogen, or boronic acid.

In some examples of Formula VIII, R¹⁸ is hydrogen. In some examples of Formula VIII, R¹⁷ is hydrogen. In some examples of Formula VIII, R¹⁸ and R¹⁷ are both hydrogen.

In some examples of Formula VIII, R¹⁶ is halogen. In some examples of Formula VIII, R¹⁶ is F. In some examples of Formula VIII, R¹⁶ is OH. In some examples of Formula VIII, R¹⁶ is H.

In some examples of Formula VIII, R¹⁵ is H. In some examples of Formula VIII, R¹⁵ is OH.

In some examples of Formula VIII, R¹⁴ is boronic acid. In some examples of Formula VIII, R¹⁴ is H.

In some examples of Formula VIII, R¹⁸ and R¹⁷ are both hydrogen and R¹⁶ is halogen. In some examples of Formula VIII, R¹⁸ and R¹⁷ are both hydrogen and R¹⁶ is F.

In some examples of Formula VIII, R¹⁸, R¹⁷, and R¹⁵ are all hydrogen. In some examples of Formula VIII, R¹⁸, R¹⁷, and R¹¹ are all hydrogen; R¹⁶ is halogen; and R¹⁴ is boronic acid. In some examples of Formula VIII, R¹⁵, R¹⁷, and R¹¹ are all hydrogen; R¹⁶ is F; and R¹⁴ is boronic acid.

In some examples of Formula VIII, R¹⁸, R¹⁷, R¹⁵, and R¹⁴ are all hydrogen. In some examples of Formula VIII, R¹⁸, R¹⁷, R¹⁵, and R¹⁴ are all hydrogen and R¹⁶ is halogen. In some examples of Formula VIII, R¹⁸, R¹⁷, R¹⁵, and R¹⁴ are all hydrogen and R¹⁶ is F.

In some examples of Formula VIII, R¹⁸, R¹⁷, and R¹⁴ are all hydrogen. In some examples of Formula VIII, R¹⁶ and R¹⁵ are both OH. In some examples of Formula VIII, R¹⁸, R¹⁷, and R¹⁴ are all hydrogen and R¹⁶ and R¹⁵ are both OH.

In some examples of Formula VIII, R¹⁸, R¹⁷, R¹⁶, R¹⁵, and R¹⁴ are all hydrogen.

In some examples of Formula VIII, R⁸, R⁹, R¹⁰, R¹¹, R¹², R¹⁴, R¹⁵, R¹⁶, R¹⁷, and R¹⁸ are each independently H, OH, halogen, boronic acid, or substituted or unsubstituted C₁-C₂₀ alkyl. In some examples of Formula VIII, R⁸, R⁹, R¹⁰, R¹¹, R¹², R¹⁴, R¹⁵, R¹⁶, R¹⁷, and R¹⁸ are each independently H, OH, halogen, boronic acid, or substituted or unsubstituted C₁-C₁₀ alkyl. In some examples of Formula VIII, R⁸, R⁹, R¹⁰, R¹¹, R¹², R¹⁴, R¹⁵, R¹⁶, R¹⁷, and R¹⁸ are each independently H, OH, halogen, boronic acid, or substituted or unsubstituted C₁-C₅ alkyl. In some examples of Formula VIII, R⁸, R⁹, R¹⁰, R¹¹, R¹², R¹⁴, R¹⁵, R¹⁶, R¹⁷, and R¹⁸ are each independently H, OH, halogen, or boronic acid. In some examples of Formula VIII, R⁸, R⁹, R¹⁰, R¹¹, R¹², R¹⁴, R¹⁵, R¹⁶, R¹⁷, and R¹⁸ are all hydrogen.

In some examples, the compound is selected from the group consisting of:

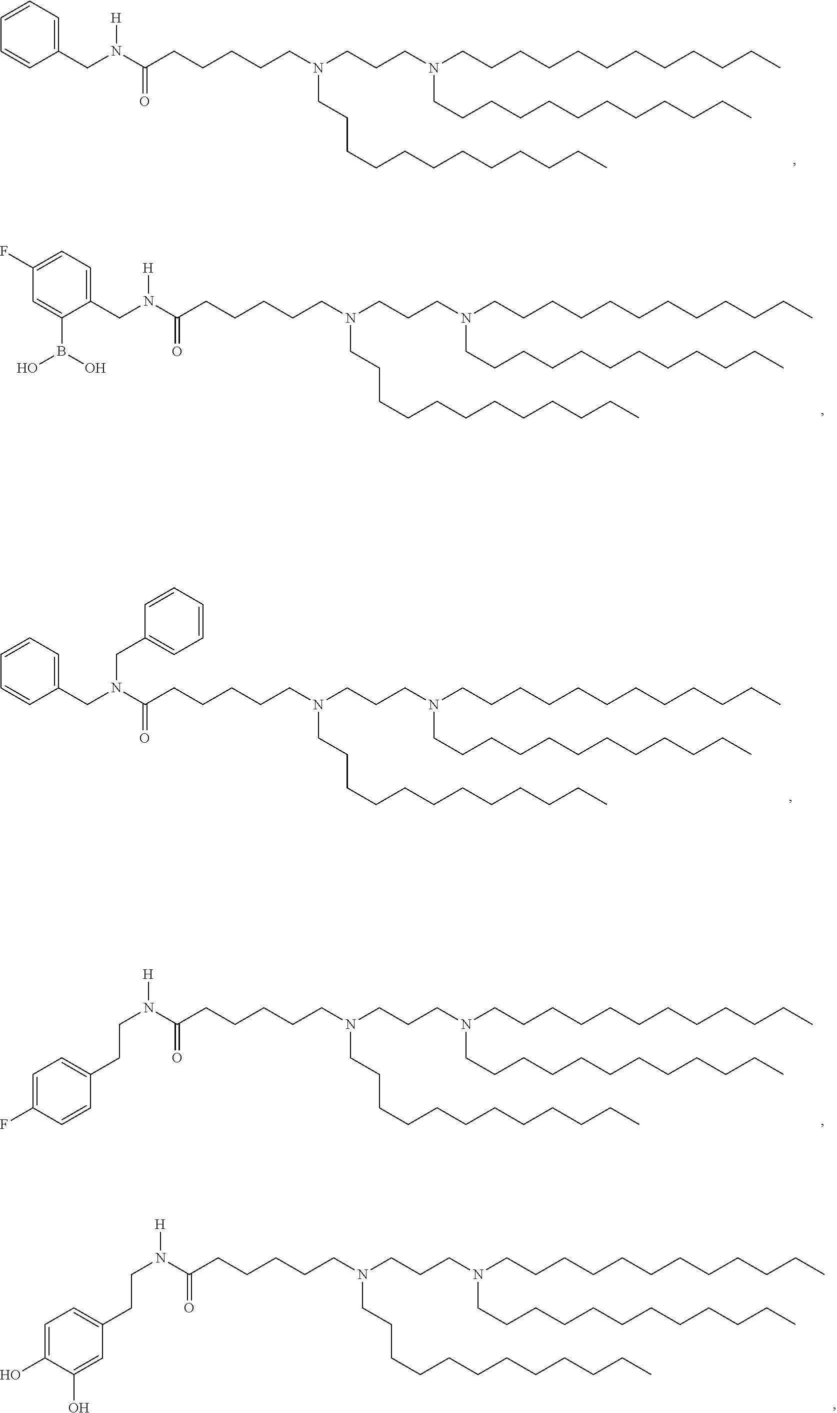 [SMILES: CCCCCCCCCCCCN(CCCCCCCCCCCC)CCCN(CCCCCCCCCCCC)CCCCCC(=O)NCc1ccccc1 ; CCCCCCCCCCCCN(CCCCCCCCCCCC)CCCN(CCCCCCCCCCCC)CCCCCC(=O)NCc1ccc(F)cc1B(O)O ; CCCCCCCCCCCCN(CCCCCCCCCCCC)CCCN(CCCCCCCCCCCC)CCCCCC(=O)N(Cc1ccccc1)Cc1ccccc1 ; CCCCCCCCCCCCN(CCCCCCCCCCCC)CCCN(CCCCCCCCCCCC)CCCCCC(=O)NCCc1ccc(F)cc1 ; CCCCCCCCCCCCN(CCCCCCCCCCCC)CCCN(CCCCCCCCCCCC)CCCCCC(=O)NCCc1ccc(O)c(O)c1]

or a pharmaceutically acceptable salt thereof.

In some examples, the compound comprises-

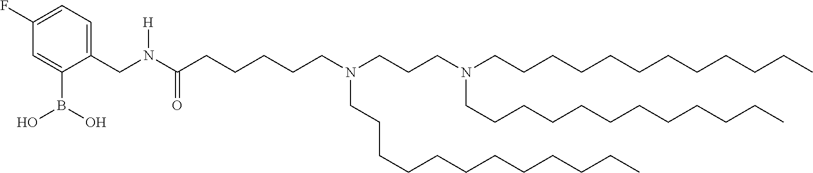 [SMILES: CCCCCCCCCCCCN(CCCCCCCCCCCC)CCCN(CCCCCCCCCCCC)CCCCCC(=O)NCc1ccc(F)cc1B(O)O]

or a pharmaceutically acceptable salt thereof.

### Lipid Particle

Also disclosed herein is a lipid particle (e.g., one or more lipid particles) comprising any of the compositions disclosed herein.

The lipid particle can be of any shape. (e.g., a sphere, a rod, a quadrilateral, an ellipse, a triangle, a polygon, etc.). In some examples, the lipid particle can have a regular shape, an irregular shape, an isotropic shape, an anisotropic shape, or a combination thereof. In some examples, the lipid particle are substantially spherical in shape.

The lipid particles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The lipid particles can, for example, have an average particle size of 50 nanometers (nm) or more (e.g., 60 nm or more, 70 nm or more, 80 nm or more, 90 nm or more, 100 nm or more, 110 nm or more, 120 nm or more, 130 nm or more, 140 nm or more, 150 nm or more, 160 nm or more, 170 nm or more, 180 nm or more, 190 nm or more, 200 nm or more, 225 nm or more, 250 nm or more, 275 nm or more, 300 nm or more, 325 nm or more, 350 nm or more, 375 nm or more, 400 nm or more, 425 nm or more, 450 nm or more, or 475 nm or more). In some examples, the lipid particles can have an average particle size of 500 nm or less (e.g., 475 nm or less, 450 nm or less, 425 nm or less, 400 nm or less, 375 nm or less, 350 nm or less, 325 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, 110 nm or less, 100 nm or less, 90 nm or less, 80 nm or less, 70 nm or less, or 60 nm or less). The average particle size of the lipid particles can range from any of the minimum values described above to any of the maximum values described above. For example, the lipid particles can have an average particle size of from 50 nm to 500 nm (e.g., from 50 nm to 275 nm, from 275 nm to 500 nm, from 50 nm to 200 nm, from 200 nm to 350 nm, from 350 nm to 500 nm, from 60 nm to 500 nm, from 50 nm to 475 nm, from 60 nm to 475 nm, from 100 nm to 200 nm, from 120 nm to 140 nm, or from 150 nm to 200 nm).

With respect to particle size distribution characterization, a parameter used to define the size range of the lipid particles is called the “polydispersity index” (PDI). The term “polydispersity” (or “dispersity” as recommended by IUPAC) is used to describe the degree of non-uniformity of a size distribution of particles. PDI is basically a representation of the distribution of size populations within a given sample. The numerical value of PDI ranges from 0.0 (for a perfectly uniform sample with respect to the particle size) to 1.0 (for a highly polydisperse sample with multiple particle size populations).

In some examples, the lipid particles can have a polydispersity index of 0.3 or less (e.g., 0.29 or less, 0.28 or less, 0.27 or less, 0.26 or less, 0.25 or less, 0.24 or less, 0.23 or less, 0.22 or less, 0.21 or less, 0.20 or less, 0.19 or less, 0.18 or less, 0.17 or less, 0.16 or less, 0.15 or less, 0.14 or less, 0.13 or less, 0.12 or less, 0.11 or less, 0.10 or less, 0.09 or less, 0.08 or less, 0.07 or less, 0.06 or less, 0.05 or less, 0.04 or less, 0.03 or less, 0.02 or less, or 0.01 or less).

In some examples, the lipid particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

In some examples, the lipid particle can further comprise an additional component, such as an additional lipid. In some examples, the additional lipid can comprise a phospholipid, a sterol, or a combination thereof. In some examples, the lipid particle can further comprise 1,2-dioleoyl-sn-glycero-3-phosphoethanolamine (DOPE), cholesterol, 1,2-dimyristoyl-rac-glycero-3-methylpolyoxyethylene, or a combination thereof.

### Pharmaceutical Compositions

Also disclosed herein are pharmaceutical compositions comprising any of the compounds or lipid particles disclosed herein.

For example, also disclosed herein are pharmaceutical compositions comprising a therapeutic agent encapsulated within any of the lipid particles disclosed herein. For example, the therapeutic agent can be encapsulated within the lipid particle with an encapsulation efficiency of 50% or more (e.g., 55% or more, 60% or more, 65% or more, 70% or more, 75% or more, 80% or more, 85% or more, 90% or more, 95% or more, or 99% or more).

The therapeutic agent can, for example, comprise an anticancer agent, an anti-inflammatory agent, an antimicrobial agent, or a combination thereof. As used herein, antimicrobials include, for example, antibacterials, antifungals, and antivirals.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, ZMapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine, and combinations thereof.

In some examples, the therapeutic agent comprises an anticancer agent. In some examples, the therapeutic agent comprises a chemotherapeutic agent, an immunotherapeutic agent, or a combination thereof.

In some examples, the therapeutic agent can comprise a chemotherapeutic agent. Chemotherapy is the treatment of cancer with one or more cytotoxic anti-neoplastic drugs (e.g., chemotherapeutic agents) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapeutic agents include, but are not limited to, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-i 1, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, Oprozomib, and combinations thereof. The anticancer agent can also include biopharmaceuticals such as, for example, antibodies.

Examples of suitable immunotherapeutic agents include, but are not limited to, alemtuzumab, cetuximab (ERBITUX), gemtuzumab, iodine 131 tositumomab, rituximab, trastuzamab (HERCEPTIN), and combinations thereof.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent comprises a nucleic acid. Particular nucleic acid examples include, but are not limited to, oligonucleotides, miRNA, shRNA, siRNA, DNA, RNA, mRNA, cDNA, double stranded nucleic acid, single stranded nucleic acid, and so forth. In a specific example, the nucleic acid can be mRNA. In some examples, the mRNA encodes a protein or peptide for therapeutic use, such as a cytokine. Examples of cytokines include, but are not limited to, IL-12, IL-27, GM-CSF, and combinations thereof.

Also disclosed herein are pharmaceutical compositions comprising nucleic acid encoding an immunotherapeutic agent encapsulated within a lipid particle. The lipid particle can comprise any suitable lipid particle. Examples of suitable lipid particles are known in the art. In some examples, the lipid particle can comprise any of the lipid particles disclosed herein. In some examples, the immunotherapeutic agent is a cytokine. Examples of cytokines include, but are not limited to, IL-12, IL-27, GM-CSF, and combinations thereof.

In some examples, the pharmaceutical composition is administered to a subject. In some examples, the subject is a mammal. In some examples, the mammal is a primate. In some examples, the mammal is a human. In some examples, the human is a patient.

In some examples, the disclosed compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

### Methods of Making

Also disclosed herein are methods of making any of the compounds or compositions disclosed herein. Also disclosed herein are methods of making any of the lipid particles disclosed herein. Also disclosed herein are methods of making any of the pharmaceutical compositions disclosed herein.

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Katchem (Prague, Czech Republic), Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical excipients disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

### Methods of Use

Also disclosed herein are methods of use of any of the compounds or compositions disclosed herein.

For example, also disclosed herein are methods of treating, preventing, or ameliorating a disease or a disorder in a subject in need thereof, the method comprising administering to the subject a therapeutically effective amount of any of the pharmaceutical compositions disclosed herein.

For example, disclosed herein are methods of treating a disease or a disorder in a subject in need thereof, the method comprising administering to the subject a therapeutically effective amount of any of the pharmaceutical compositions disclosed herein.

Examples of diseases and disorders include, but are not limited to, cancer, inflammatory diseases, neurodegenerative diseases, psychotropic disorders, autoimmune diseases, genetic diseases, infectious diseases, and combinations thereof.

In some examples, the disease comprises cancer. For example, the compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or may be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can, in some examples, further include treatment with one or more additional agents (e.g., an anti-cancer agent or ionizing radiation). For example, the compounds or compositions or pharmaceutically acceptable salts thereof as described herein can be combined into a pharmaceutical composition with an additional anticancer agent. The additional anti-cancer agent can also include biopharmaceuticals such as, for example, antibodies. Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of suppressing tumor growth in a subject. The method includes contacting at least a portion of the tumor with a therapeutically effective amount of any of the compound or compositions as described herein. In some examples, the methods further include the step of irradiating at least a portion of the tumor with a therapeutically effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. A therapeutically effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

In some examples, the diseases comprises an inflammatory disease. Inflammatory diseases include, but are not limited to, acne vulgaris, ankylosing spondylitis, asthma, autoimmune diseases, Celiac disease, chronic prostatitis, Crohn's disease, glomerulonephritis, hidradenitis suppurativa, inflammatory bowel diseases, pelvic inflammatory disease, psoriasis, reperfusion injury, rheumatoid arthritis, sarcoidosis, vasculitis, interstitial cystitis, type 1 hypersensitivities, systemic sclerosis, dermatomyositis, polymyositis, and inclusion body myositis.

In some examples, the disease comprises a neurodegenerative disease. Neurodegenerative diseases include, but are not limited to, Alzheimer's disease, amyotrophic lateral sclerosis (ALS), Alpers' disease, batten disease, Benson's syndrome, Cerebro-oculo-facio-skeletal (COFS) syndrome, corticobasal degeneration, Creutzfeldt-Jakob disease, dementias, Friedreich's ataxia, Gerstmann-Strussler-Scheinker disease, Huntington's disease, Lewy body syndrome, Leigh's disease, monomelic amyotrophy, motor neuron diseases, multiple system atrophy, opsoclonus myoclonus, progressive multifocal leukoencephalopathy, Parkinson's disease, Prion diseases, primary progressive aphasia, progressive supranuclear palsy, spinocerebellar ataxia, spinal muscular atrophy, kuru, and Shy-Drager syndrome.

In some examples, the disease comprises a psychotropic disorder. Psychotropic disorders include, but are not limited to, attention deficit disorder (ADD), attention deficit hyperactive disorder (ADHD), anorexia nervosa, anxiety, dipolar disorder, bulimia, depression, insomnia, neuropathic pain, mania, obsessive compulsive disorder (OCD), panic disorder, premenstrual dysphoric disorder (PMDD), mood disorder, serotonin syndrome, schizophrenia, and seasonal affective disorder.

The methods of treatment of the disease or disorder described herein can further include treatment with one or more additional agents. The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

In some examples, the compound or composition can be administered to the subject in an amount of 1 microgram (pg) per kilogram (kg) of body weight of the subject per day (pg/kg/day) or more (e.g., 2 μg/kg/day or more, 3 μg/kg/day or more, 4 μg/kg/day or more, 5 μg/kg/day or more, 10 μg/kg/day or more, 15 μg/kg/day or more, 20 μg/kg/day or more, 25 μg/kg/day or more, 30 μg/kg/day or more, 35 μg/kg/day or more, 40 μg/kg/day or more, 45 μg/kg/day or more, 50 μg/kg/day or more, 60 μg/kg/day or more, 70 μg/kg/day or more, 80 μg/kg/day or more, 90 μg/kg/day or more, 100 μg/kg/day or more, 125 μg/kg/day or more, 150 μg/kg/day or more, 175 μg/kg/day or more, 200 μg/kg/day or more, 225 μg/kg/day or more, 250 μg/kg/day or more, 300 μg/kg/day or more, 350 μg/kg/day or more, 400 μg/kg/day or more, 450 μg/kg/day or more, 500 μg/kg/day or more, 600 μg/kg/day or more, 700 μg/kg/day or more, 800 μg/kg/day or more, 900 μg/kg/day or more, 1 milligram (mg)/kg/day or more, 2 mg/kg/day or more, 3 mg/kg/day or more, 4 mg/kg/day or more, 5 mg/kg/day or more, 6 mg/kg/day or more, 7 mg/kg/day or more, 8 mg/kg/day or more, or 9 mg/kg/day or more). In some examples, the compound or composition can be administered to the subject in an amount of 10 milligrams (mg) per kilogram (kg) of body weight of the subject per day (mg/kg/day) or less (e.g., 9 mg/kg/day or less, 8 mg/kg/day or less, 7 mg/kg/day or less, 6 mg/kg/day or less, 5 mg/kg/day or less, 4 mg/kg/day or less, 3 mg/kg/day or less, 2 mg/kg/day or less, 1 mg/kg/day or less, 900 μg/kg/day or less, 800 μg/kg/day or less, 700 μg/kg/day or less, 600 μg/kg/day or less, 500 μg/kg/day or less, 450 μg/kg/day or less, 400 μg/kg/day or less, 350 μg/kg/day or less, 300 μg/kg/day or less, 250 μg/kg/day or less, 225 μg/kg/day or less, 200 μg/kg/day or less, 175 μg/kg/day or less, 150 μg/kg/day or less, 125 μg/kg/day or less, 100 μg/kg/day or less, 90 μg/kg/day or less, 80 μg/kg/day or less, 70 μg/kg/day or less, 60 μg/kg/day or less, 50 μg/kg/day or less, 45 μg/kg/day or less, 40 μg/kg/day or less, 35 μg/kg/day or less, 30 μg/kg/day or less, 25 μg/kg/day or less, 20 μg/kg/day or less, 15 μg/kg/day or less, 10 μg/kg/day or less, 5 μg/kg/day or less, 4 μg/kg/day or less, 3 μg/kg/day or less, or 2 μg/kg/day or less).

The amount of the compound or composition administered to the subject can range from any of the minimum values described above to any of the maximum values described above. For example, the compound or composition can be administered to the subject in an amount of from 1 microgram (pg) per kilogram (kg) of body weight of the subject per day to 10 milligrams (mg)/kg/day (e.g., from 1 μg/kg/day to 100 μg/kg/day, from 100 μg/kg/day to 10 mg/kg/day, from 1 μg/kg/day to 10 μg/kg/day, from 10 μg/kg/day to 100 μg/kg/day, from 100 μg/kg/day to 1 mg/kg/day, from 1 mg/kg/day to 10 mg/kg/day, from 5 μg/kg/day to 10 mg/kg/day, from 1 μg/kg/day to 5 mg/kg/day, or from 5 to 5 mg/kg/day).

It is understood, however, that the specific dose level for any particular subject will depend upon a variety of factors. Such factors include the age, body weight, general health, sex, and diet of the subject. Other factors include the time and route of administration, rate of excretion, drug combination, and the type and severity of the particular disease or disorder.

The methods, compounds, and compositions as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

In certain embodiments, it is desirable to target a nanoparticle using a targeting moiety that is specific to a cell type and/or tissue type. In some embodiments, a nanoparticle may be targeted to a particular cell, tissue, and/or organ using a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5):439-62, 2003 and Abra et al., J. Liposome Res. 12:1-3, 2002.

The targeting moiety can target any known cell type, including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte. In other embodiments, the targeting moiety targets the lipid nanoparticle to a colon cell. In some embodiments, the targeting moiety targets the lipid nanoparticle to a liver cancer cell (e.g., a hepatocellular carcinoma cell) or a colorectal cancer cell (e.g., a primary tumor or a metastasis).

### Compositions, Formulations, Methods of Administration, and Kits

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable excipient in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and application. The compositions can also include conventional pharmaceutically-acceptable carriers and diluents which are known to those skilled in the art.

Examples of carriers or diluents for use with the compounds include ethanol, dimethyl sulfoxide, glycerol, alumina, starch, saline, and equivalent carriers and diluents. To provide for the administration of such dosages for the desired application, compositions disclosed herein can comprise between about 0.1% and 100% by weight of the total of one or more of the subject compounds based on the weight of the total composition including carrier or diluent.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art.

For the treatment of oncological disorders, the compounds or compositions disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds or compositions disclosed herein. For example, the compounds or compositions disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Pharmaceutical compositions disclosed herein suitable for injectable use include sterile aqueous solutions or dispersions. Furthermore, the compositions can be in the form of sterile powders for the extemporaneous preparation of such sterile injectable solutions or dispersions. In some examples, the final injectable form can be sterile and can be effectively fluid for easy syringability. In some examples, the pharmaceutical compositions can be stable under the conditions of manufacture and storage; thus, they can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions disclosed herein can be in a form suitable for topical use such as, for example, an aerosol, cream, ointment, lotion, dusting powder, mouth washes, gargles, solution, tincture, and the like. In some examples, the compositions can be in a form suitable for use in transdermal devices. In some examples, it will be desirable to administer them topically to the skin as compositions, in combination with a dermatologically acceptable carrier, which can be a solid or a liquid. Compounds and agents and compositions disclosed herein can be applied topically to a subject's skin. These formulations can be prepared, utilizing any of the compounds disclosed herein or pharmaceutically acceptable salts thereof, via conventional processing methods.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Pharmaceutical compositions disclosed herein can be in a form suitable for rectal administration wherein the carrier is a solid. In some examples, the mixture forms unit dose suppositories. Suitable carriers include cocoa butter and other materials commonly used in the art. The suppositories can be conveniently formed by first admixing the composition with the softened or melted carriers) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing any of the compounds disclosed herein, and/or pharmaceutically acceptable salts thereof, can also be prepared in powder or liquid concentrate form.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

In some examples, the kit further comprises at least one agent, wherein the compound and the agent are co-formulated.

In some examples, the compound and the agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Effective Inhibition of Tumor Growth by Intratumoral Delivery of IL-12 and IL-27 Using Diamino Lipid (DAL) Nanoparticles

### Abstract:

Cytokines are important immunotherapeutics with approved drugs for the treatment of human cancers. However, systemic therapy using cytokines often fail to achieve adequate concentrations at immune cells in the tumor due to dose-limiting toxicity. Thus, developing localized therapy that directly delivers immune stimulatory cytokines to tumors can improve the therapeutic efficacy. Disclosed herein are the anti-tumor effects of lipid nanoparticles (LNPs) that deliver cytokine mRNAs including IL-12, IL-27 and GM-CSF to tumors. Ionizable lipid materials containing di-amino groups were synthesized with various head groups (DALs). Then, the ionizable lipid was formulated with phospholipid, cholesterol, and PEG-lipid to encapsulate single or multiple cytokine mRNAs. The formulation was effective in delivering mRNAs in vitro and in vivo. Additionally, it was found that intratumoral delivery of DAL4-LNP loaded with IL-12 mRNA was most effective in inhibiting B16 melanoma tumor growth compared to IL-27 or GM-CSF mRNAs LNPs in monotherapy. Moreover, intratumoral delivery of IL-12 mRNA and IL-27 mRNA by DAL4-LNPs showed a synergistic effect in inhibiting tumor growth. Thus, intratumoral administration of IL12 and IL27 mRNA LNPs provides a new treatment strategy for cancer treatment.

### Introduction:

Cytokines are one of the first immunotherapeutics applied for treating human cancers. IFN-α was the first cytokine drug approved by the FDA for the treatment of hairy cell leukemia back in 19861, and IL-2 was later approved for metastatic renal cell cancer and advanced melanoma in 1992 and 1998, respectively². Many cytokines are currently being studied in clinical trials, including IL-12, IL-15, IL-21 and GM-CSF³. Despite efforts to develop systemic cytokine monotherapy for cancers, the delivery of cytokines hinders the initial excitement due to a number of reasons. First, soluble cytokines are often released in response to stimulus and act in a paracrine or autocrine fashion due to their short half-life. Therefore, large quantities of cytokines must be administered to achieve a sufficient concentration within the tumor, which is often associated with severe toxicities⁴⁻⁶. Second, due to dose-limiting toxicities, lower doses of cytokines are often used in clinical trials, which makes it difficult to reach adequate concentrations in the tumor microenvironment (TME). Thus, developing localized therapy that directly delivers immune stimulatory cytokines to tumors may be a promising strategy to overcome these issues.

IL-12, IL-27 and GM-CSF have shown anti-tumor activity in animal models. IL-12 demonstrates potent anti-tumor activity⁷ via enhanced Th1/Tc1 response^{7, 8} and T cell recruitment to tumors⁹. However, systemically delivered IL-12 can lead to fatal consequences^{5, 6} To avoid systemic toxicity, researchers have designed various strategies¹⁰⁻¹⁶ to target IL-12 in tumors. However, IL-12-induced systemic toxicity can only be relieved if IL-12 expression is confined to the tumor site¹⁷. Mice inoculated with live or irradiated tumor cells expressing high levels of GM-CSF (GVAX) resulted in the recruitment of antigen-presenting cells such as dendritic cells in tumor sites, which lead to induction of anti-tumor immunity and tumor rejection^{18, 19} The efficacy of autologous and allogeneic GVAX was later reported either as a single drug or in combination with other immunomodulators in various animal models and clinical studies²⁰⁻²². GM-CSF has also been included in two clinically approved anti-tumor vaccines, i.e. Sipuleucel-T²³ and T-VEC²⁴. Systemically delivered GM-CSF has shown potential clinical benefits when used in combination with ipilimumab^{25, 26}, and interestingly, GM-CSF was shown to be associated with reduced toxicity caused by ipilimumab. IL-27 is an anti-inflammatory cytokine that exhibits potent anti-tumor activity. Animal studies from have indicated that both endogenous²⁷⁻³⁰ and exogenous³¹⁻³³ IL-27 inhibit tumor growth. It was found that IL-27 enhances T cell survival in TME and promotes the generation of memory T cells by programming CD8⁺ T cells into a unique T effector phenotype, characterized by increased secretion of IFN-γ and IL-10^{31, 34}.

Systemic toxicity of cytokines and potent tumor local effects in induction of anti-tumor immunity strongly justify the development of clinically relevant, cytokine-based local therapy. It has been found that intratumoral delivery of AAV-IL-27 could inhibit tumor growth and induced anti-tumor immunity³⁵. However, AAV-mediated IL-27 delivery poses a potential problem as there is no termination of IL-27 production when the biological activity of IL-27 is sufficient. Therefore, approaches that can efficiently deliver immunostimulatory cytokines to tumors are necessary. In this context, disclosed herein is the use of lipid nanoparticles to deliver mRNAs of immune stimuli to TME is a highly feasible approach. LNPs possess unique features including: (i) easy preparation and chemical synthesis for large-scale production; (ii) efficient encapsulation and delivery of mRNA; (iii) transient, mRNA-induced expression of protein; and (iv) low potential toxicity due to tumor local application.

Shown herein is the local delivery of LNPs encapsulating cytokine mRNA combination that has not been tested before in a mouse melanoma model. The formulation was able to deliver cytokine mRNAs in vitro and in vivo. Moreover, intratumoral administration of IL12+IL27 mRNA by LNPs induced sustained inhibition of B16F10 melanoma growth. In summary, in this study, a new mRNA delivery formulation and a cytokine combination is shown that can be used for improving the current cancer treatment. This can be used in combination with multiple cancer therapeutics.

### Results

### Design and Synthesis of Diaimino Lipid Derivatives:

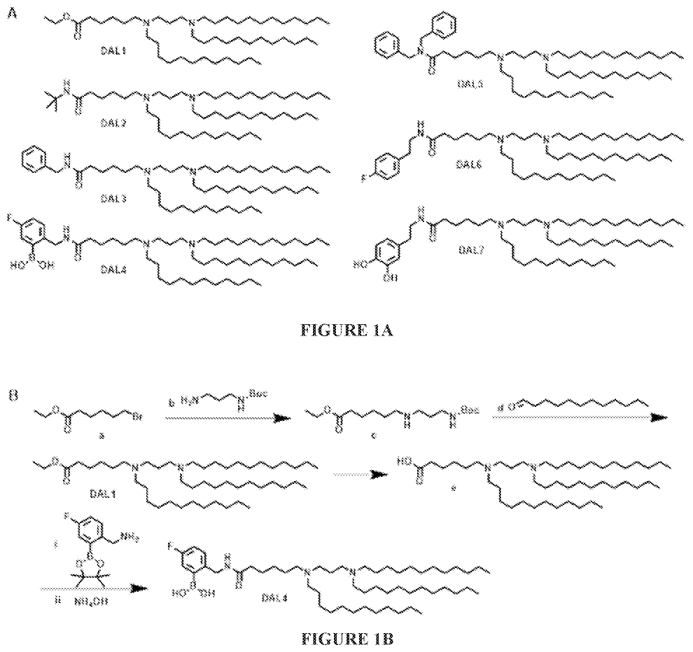

A library of seven diamino lipid materials (DALs) was designed and synthesized with the same diamino core and carbon chains but with seven different head groups (FIG. 1A). The tertiary amines in the diamino core can be ionized to interact with mRNA at acidic pH. FIG. 1B displays a representative synthetic route. Compound a underwent a substitution reaction with b to give c. Subsequently, a reductive amination reaction between c and d produced DALL. Removal of ethyl group on DAL1 yielded e, which was further reacted with 5-Fluoro-2-aminomethylphenylboronic acid, pinacol ester and then was hydrolyzed to afford DA LA. The structures of all these DALs using both ¹H NMR and mass spectrum (MS) are shown here:

### Synthesis of DALs

Synthesis of DAL2: yield 37%. ¹H NMR (400 MHz, CDCl₃): δ=2.45-2.39 (11H, m), 2.11-2.07 (2H, m), 1.64-1.57 (2H, m), 1.46-1.42 (6H, m), 1.35 (9H, s), 1.27 (59H, m), 0.91-0.87 (9H, t, J=8). MS (m/z): [M+H]⁺ calcd. for C₄₉H₁₀₂N₃O, 748.8023; found, 748.8022.

Synthesis of DAL3: yield 61%. ¹H NMR (400 MHz, CDCl₃): δ=7.37-7.27 (5H, m), 4.47-4.46 (2H, m), 2.43-2.38 (11H, m), 2.25-2.22 (2H, m), 1.72-1.68 (2H, m), 1.58 (2H, m), 1.47-1.43 (8H, m), 1.28 (57H, m), 0.92-0.88 (9H, t, J=8). MS (m/z): [M+H]⁺ calcd. for C₅₂H₁₀₀N₃O, 782.7866; found, 1529.0320.

Synthesis of DAL4: yield 33%. ¹H NMR (400 MHz, CDCl₃): δ=7.68-7.66 (1H, m), 7.36 (1H, s), 6.94-6.83 (1H, m), 4.70-4.41 (2H, m), 2.62-2.45 (1OH, m), 2.00 (1H, s), 1.62-1.28 (59H, m), 0.90-0.86 (9H, t, J=8). MS (m/z): [M+H]⁺ calcd. for C₅₂H₁₀₀BFN₃O₃, 844.7842, found, 844.7852; [M+CH₂+H]⁺ calcd. for C₅₃H₁₀₂BFN₃O₃, 858.7998, found, 858.8007; [M+2CH₂+H]⁺ calcd. for C₅₄H₁₀₄BFN₃O₃, 872.8165, found, 872.8161. (Note: The amine used for synthesizing DAL4 was 5-Fluoro-2-aminomethylphenylboronic acid, pinacol ester. The pinacol ester was hydrolyzed during purification through column chromatography to yield pure DAL4.)

Synthesis of DALS: yield 19%. ¹H NMR (400 MHz, CDCl₃): δ=7.36-7.30 (6H, m), 7.25-7.16 (4H, m), 4.62 (2H, s), 4.47 (2H, s), 2.46-2.41 (13H, m), 1.77-1.73 (2H, m), 1.59 (2H, s), 1.44-1.43 (9H, m), 1.28 (57H, s), 0.92-0.89 (9H, t, J=8). MS (m/z): [M+H] calcd. for C₅₉H₁₀₆N₃O, 872.8336; found, 872.8313.

Synthesis of DAL6: yield 78%. ¹H NMR (400 MHz, CDCl₃): δ=7.17-7.14 (2H, m), 7.02-6.98 (2H, m), 3.52-3.47 (2H, m), 2.82-2.78 (2H, m), 2.42-2.37 (11H, m), 2.15-2.11 (2H, t, J=8), 1.64-1.55 (1H, m), 1.44-1.40 (5H, m), 1.27 (61H, m), 0.91-0.87 (9H, t, J=8). MS (m/z): [M+H]⁺ calcd. for C₅₃H₁₀₁FN₃O, 814.7928; found, 814.7922.

Synthesis of DAL7: yield 58%. ¹H NMR (400 MHz, CDCl₃): δ=6.80-6.78 (1H, m), 6.56-6.49 (2H, m), 3.51 (2H, m), 2.70-2.67 (2H, m), 2.54-2.43 (11H, m), 2.15-2.12 (2H, m), 1.68 (2H, m), 1.60-1.56 (2H, m), 1.47-42 (8H, m), 1.238 (56H, m), 0.92-0.89 (9H, t, J=8). MS (m/z): [M+H]⁺ calcd. for C₅₃H₁₀₂N₃O₃, 828.7921; found, 828.7887.

Formulation Screening and In Vitro mRNA Delivery Using DAL Lipid Nanoparticles

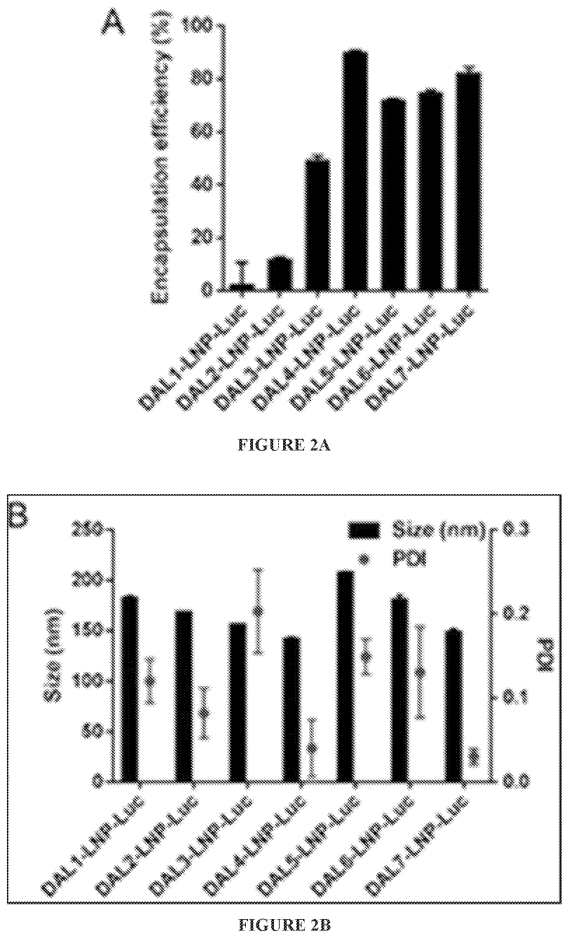

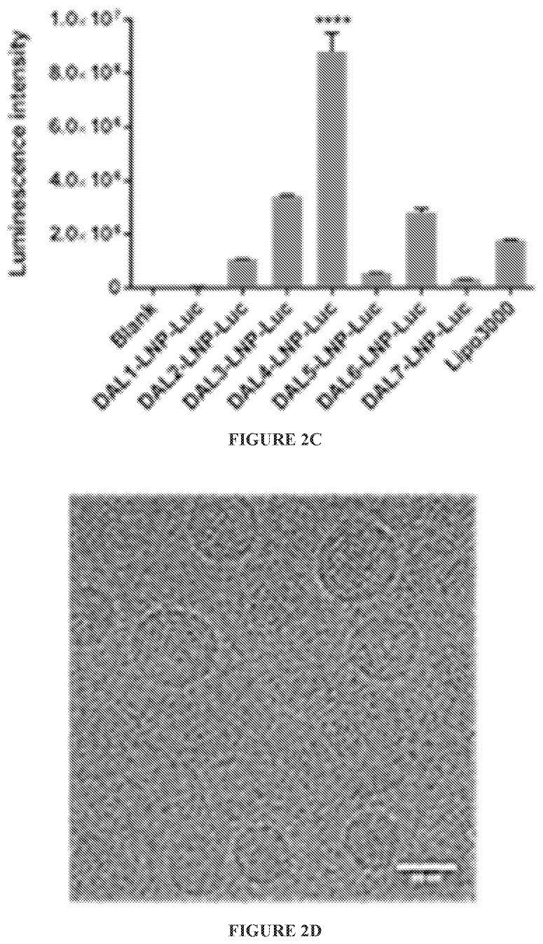

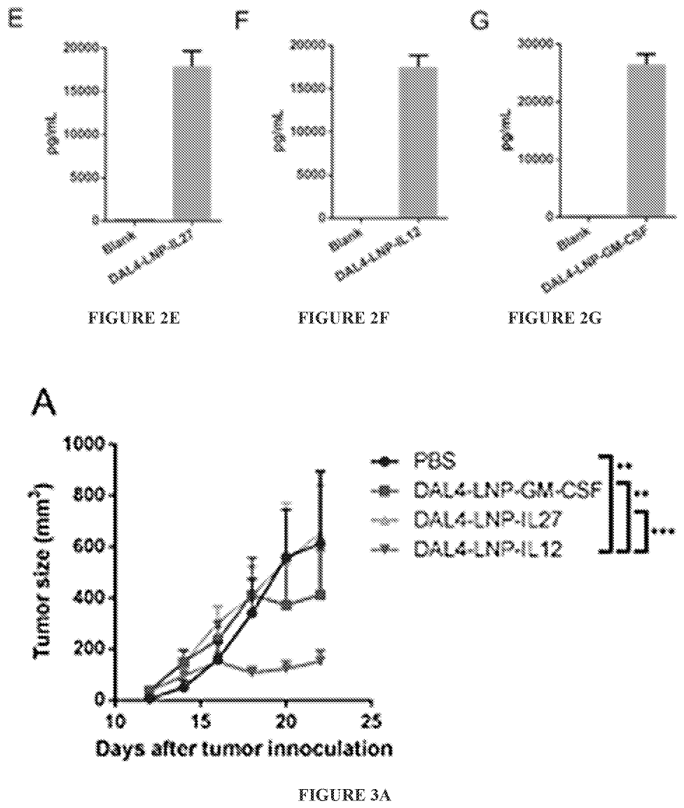

Next, the mRNA delivery efficiency of DAL lipid nanoparticles (DAL-LNPs) was studied using luciferase mRNA in B16 melanoma tumor cells in vitro. DALs were formulated with 1,2-dioleoylsnglycero- 3-phosphoethanolamine (DOPE), cholesterol (Chol), 1,2-dimyristoyl-rac-glycero-3-methylpolyoxyethylene (DMG-PEG₂₀₀₀, PEG) (molar ratio: DAL/DOPE/Chol/PEG=20/30/40/0.75), and luciferase mRNA to prepare the nanoparticles (DAL-LNP-Luc) as described previously³⁶. The mRNA encapsulation efficiency (FIG. 2A), formulation size distribution (FIG. 2B) and polydispersity index (PDI) (FIG. 2B) were also determined. The nanoparticles had a typical size of about 150-200 nm in diameter (FIG. 2B). After treating the B16 cells with DAL-LNPs for 18 hr, the luciferase mRNA delivery efficiency was determined by a bioluminescence reporter assay. The results shown in FIG. 2C suggested that DAL4-LNPs induced the highest luminescence signal in cultured cells when compared with other DAL-LNPs and Lipofectamine™ 3000-based formulations. DAL4-LNPs showed spherical morphology visualized by cryo-EM microscopy (FIG. 2D). Thus, DAL4-LNPs was chosen to encapsulate cytokine mRNAs for further testing.

The cytokine mRNA delivery and protein expression in vitro was checked by ELISA assay. The B16 cells were treated with DAL4-LNPs encapsulating either IL-27 mRNA (DAL4-LNP-IL27), IL-12 mRNA (DAL4-LNP-IL12), or GM-CSF mRNA (DAL4-LNP-GM-CSF) for 18 hr and collected culture supernatants. The ELISA results (FIG. 2E-G) showed that each of the cytokine proteins can be expressed and secreted in the supernatants of cell culture, indicating that the DAL4-LNPs can effectively deliver cytokine mRNA in cells.

In Vivo Anti-Cancer Efficacy of DAL Cytokine mRNA LNPs

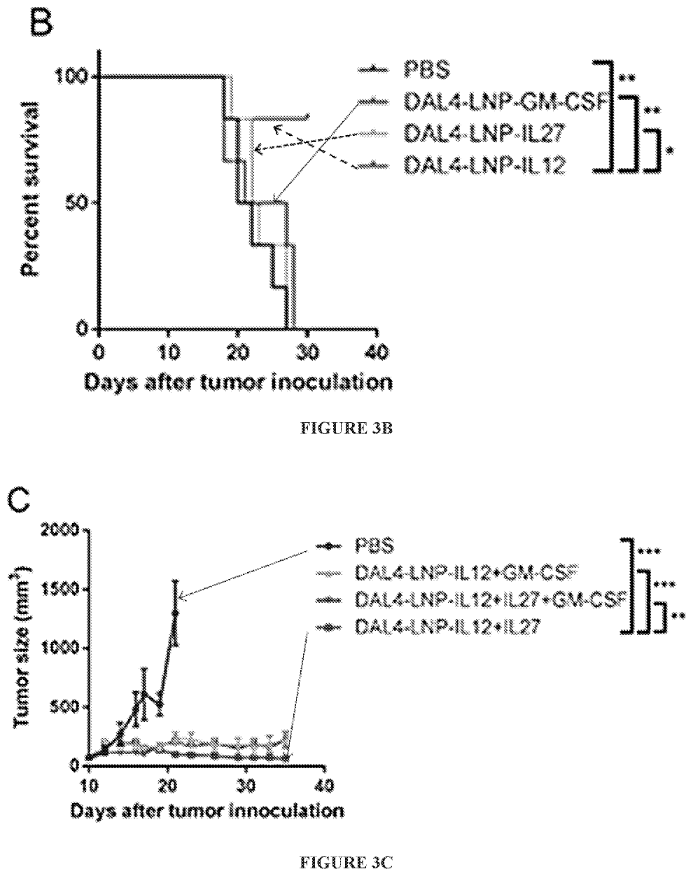

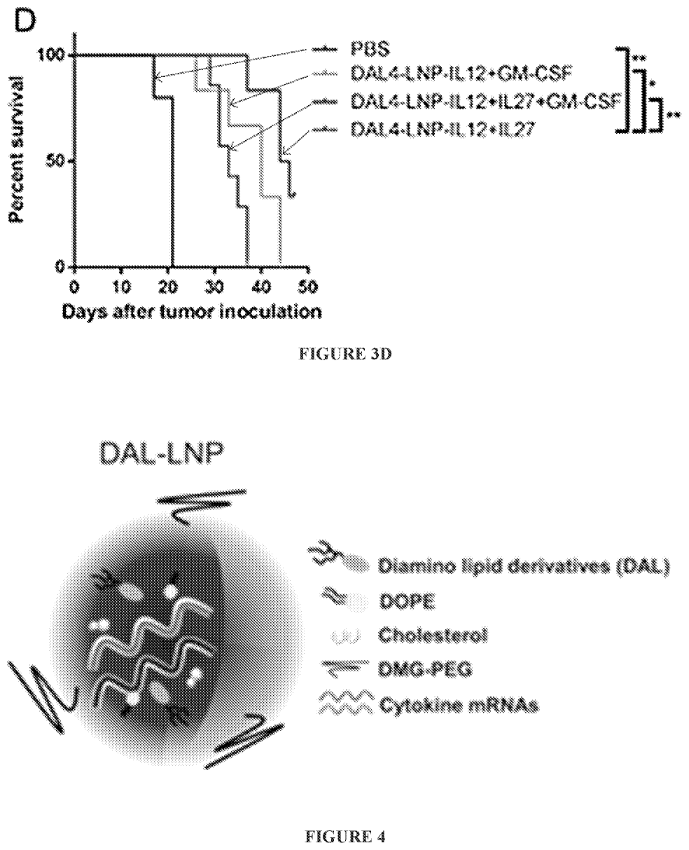

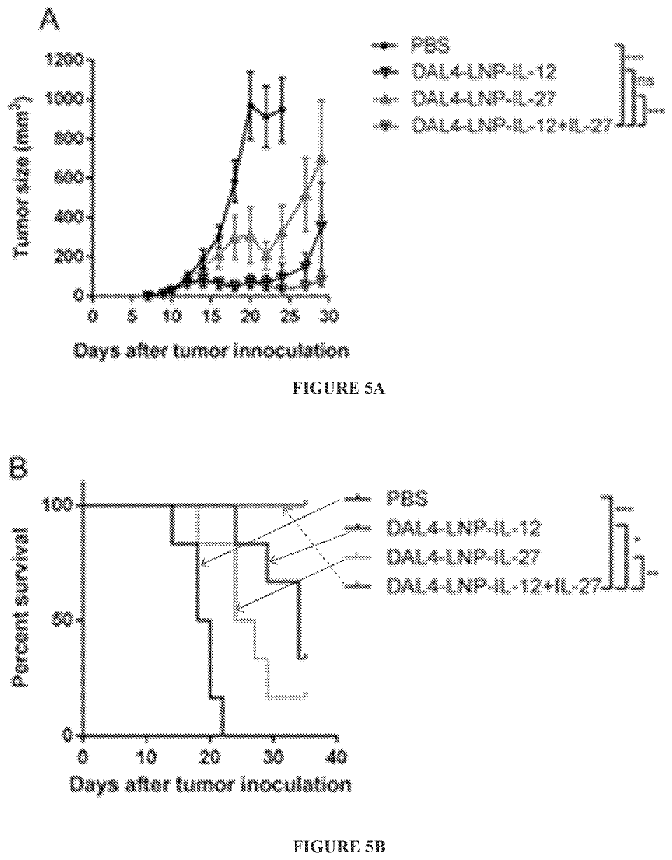

Cytokines such as IL-12⁷, IL-27³¹⁻³³ and GM-CSF^{18,19} have demonstrated anti-tumor activity when delivered systemically or locally. To compare the therapeutic potential of each DAL-LNP cytokine mRNA formulation, the treatment efficacy was first studied in a subcutaneous B16 mouse tumor model by delivering single cytokine mRNA with DAL4-LNP via intratumoral injection. DAL4-LNPs loaded with IL12 mRNA (DAL4-LNP-IL12) showed a stronger tumor inhibitive effect when compared to DAL4-LNP encapsulated with IL-27 mRNA (DAL4-LNP-IL27) or GM-CSF mRNA (DAL4-LNP-GM-CSF) based on the data of tumor size and mice survival (FIG. 3A-B).

Gene therapy based on the systemic application of IL-27 and IL-12 has shown significant synergy of these two cytokines in tumor inhibition³⁷. To determine if intratumoral injections of cytokine mRNA nanoparticles could induce stronger tumor growth inhibition, different combinations of cytokine mRNAs in DAL4-LNPs were tested. As shown in FIG. 5, FIG. 3C, and FIG. 3D, DAL4-LNP-IL 12+IL27 outperformed other single cytokine or combination of cytokines by inhibiting tumor growth (FIG. 3C) and extending survival of tumor-bearing mice (FIG. 3D). Interestingly, the combination of all three cytokine mRNAs did not induce better tumor inhibition compared to the IL-12+IL-27 combination.

Discussion In this study, the strategy of using lipid nanoparticles to deliver cytokine mRNAs to TME was tested. Seven new ionizable lipids containing di-amino groups and various head groups were synthesized. Next, these ionizable lipids were formulated with phospholipid, cholesterol, and PEG-lipid to encapsulate luciferase mRNA or single or multiple cytokine mRNAs. The mRNA was screened and characterized for formulation by delivering luciferase mRNA in B16F10 melanoma cells in vitro. DAL4-LNPs showed the highest luciferase mRNA delivery efficiency with a size of ˜130 nm and mRNA encapsulation efficiency of around 90%. Furthermore, cytokine mRNAs, including IL12, GM-CSF, and IL27 mRNA, were encapsulated in DAL4-LNPs and tested the in vitro delivery efficiency in cultured B16F10 melanoma cells. The ELISA assay detected the secreted cytokines in the supernatant of the cultured cells, indicating the DAL4-LNPs could deliver cytokines mRNA in cancer cells. The in vivo anti-cancer efficacy of DAL4-LNPs loaded with cytokine mRNAs were then tested in B16F10 mouse melanoma model. The therapeutic efficacy of DAL4-LNPs carrying single cytokine mRNA was tested. It was found that DAL4-LNP-IL12 was most potent as compared to DAL4-LNP-IL27 or DAL4-LNP-GM-CSF. To further study the cytokine combination effects, two or three cytokine mRNAs were co-encapsulated in DAL4-LNPs and their anti-cancer efficacy was explored via intratumoral injection. The results revealed that DAL4-LNP-IL12+IL27 outperformed single cytokine or other combinations of cytokines with stronger tumor inhibition and prolonged survival of tumor-bearing mice.

This approach of cytokine tumor local delivery suggests IL-12 is an effective anti-tumor agent, which is consistent with previous studies that IL-12 promotes Th1/Tc1 response^{7, 8} and enhances T cell trafficking to tumors⁹. As a member of the IL-12 cytokine family, IL-27 promotes similar anti-tumor effects as IL-12^{38, 39}. Despite similar outcomes, IL-12 and IL-27 activate T cells and NK cells through different pathways: IL-12 via Stat4 and IL-27 through Stat1 and Stat3. This explains the synergistic effect of these two cytokines in systemic therapy³⁷. In this study, it was also shown that local therapy using DAL4-LNP-IL 12+IL27 had strong synergy.

A notable observation in this study is that the addition of GM-CSF does not further improve the therapeutic efficacy of IL-12 and IL-27 mRNA LNPs. It is possible that less effectiveness may be related to GM-CSF-mediated expansion of myeloid-derived suppressor cells^{40, 41}, which could inhibit IL-12/IL-27-mediated anti-tumor immune responses and promote tumor growth.

Taken together, disclosed herein is a new mRNA delivery formulation and a cytokine combination that can be used for improving the current cancer treatment. Such a delivery platform merits further development for testing more immunotherapeutics for cancer.

### Materials and Methods

### Reagents:

All chemicals were purchased from Sigma-Aldrich (St. Louis, MO, USA), except DOPE was purchased from Avanti Polar Lipids, Inc (Alabaster, AL, USA) and DMG-PEG₂₀₀₀ from NOF America Corporation (White Plains, NY, USA).

### Synthesis of DALs:

Compounds b, c and DAL1 were synthesized according to methods reported previously³⁶.

Synthesis of DAL2: DAL1 (250 mg, 0.35 mmol) was first hydrolyzed by NaOH aqueous (1 M) in a mixture of THF and MeOH at 70° C. for 3 h. Next, 100 mL CH₂Cl₂ was added, which was dried with MgSO₄. Then, the solvent was evaporated and 10 mL anhydrous THE was added to the residue. To the solution was added NHS (125 mg, 1.05 mmole) and DCC (230 mg, 1.05 mmole). The resulting mixture was stirred at room temperature overnight. tert-butylamine (80 mg, 1.05 mmole) and TEA (150 μL, 1.2 mmole) were added to the above reacting mixture. The resulting mixture was stirred at room temperature overnight. After the solvent was removed under reduced temperature, the residue was purified by column chromatography using a CombiFlash Rf system with a RediSep Gold Resolution silica column (Teledyne Isco) with gradient elution (CH₂Cl₂ and ultra) from 100% CH₂Cl₂ to 80% CH₂Cl₂ (ultra, CH₂Cl₂/MeOH/NH₄OH=75/22/3 by volume) to give 96 mg oil like DAL2. DAL3-7 were synthesized following the same procedure as used for the synthesis of DAL2. The amine used for synthesizing DAL4 was 5-Fluoro-2-aminomethylphenylboronic acid, pinacol ester. The pinacol ester was hydrolyzed during purification through column chromatography to yield pure DAL4.

mRNA Synthesis:

IL-12, IL-27, and GM-CSF plasmids were purchased from InvivoGen (San Diego, CA, USA) and were amplified to generate templates for in vitro transcription. mRNA transcripts were synthesized as reported previously^{36, 42}. mRNAs were synthesized with full substitution of UTP by pseudouridine-5′-triphosphate (TriLink, USA) using AmpliScribe T7-Flash Transcription Kit (Lucigen, USA) following the manufacturer's instruction. The resulting mRNAs were then purified by RNA Clean & Concentrator (Zymo, USA) and capped using Vaccinia Capping System (NEB, USA) and Cap 2′-O-Methyltransferase (NEB, USA). After one final round of purification, mRNA concentrations were measured using a NanoDrop 2000 Spectrophotometer (ThermoFisher, USA) and stored at −80° C. for future use.

Preparation and Characterization of mRNA LNPs:

mRNA NPs were prepared by mixing lipid materials dissolved in ethanol and mRNA solution diluted in citrate buffer (pH=3)⁴³. In vitro used NPs were prepared by the pipetting method. In vivo used NPs were prepared by the microfluidic device (Precision NanoSystems, Vancouver, BC, Canada) and dialyzed in PBS (Slide-A-Lyzer™ Dialysis Cassettes, 3.5K MWCO, ThermoFisher). The size and zeta potential of DAL4-LNPs were measured by Zetasizer (Malvern, MA, USA). The mRNA encapsulation efficiency (EE %) was determined by the RiboGreen assay. Cryo-EM image of DAL4-LNP-IL12+IL27 was obtained by Glacios Cryo Transmission Electron Microscope (ThermoFisher) using a similar method reported before³⁶.

In Vitro Delivery of Cytokine mRNAs LNP to B16F10 Melanoma Cells:

B16F 10 cells were originally purchased from ATCC and were maintained in the lab. To check the delivery of DAL4-LNPs encapsulating cytokine mRNAs, cultured B16 cells were treated with DAL4-LNP-IL 12, DAL4-LNP-IL27, DAL4-LNP-GM-CSF respectively, with a dose of 50 ng mRNA of each cytokine mRNA. After 18 hr incubation, the supernatants of cell cultures were collected and tested by ELISA kit following standard procedures.

In Vivo Anti-Cancer Efficacy of Cytokine mRNA LNPs:

All animal experiments were performed in accordance with the Guidelines for Care and Use of Laboratory Animals of The Ohio State University and were approved by the Animal Ethics Committee of Institutional Animal Care and Use Committee (IACUC). To set up the mice tumor model, 1×10⁵ B16F10 cells in 100 μL PBS were subcutaneously injected in C57BL/6 mice. Mice were randomly grouped (n=5-7 in each group) when the tumor reached about 50 mm³. Intratumoral treatment with cytokine mRNA LNPs (2 μg per mRNA/injection) was performed every other day for six times in total. The tumor volume is calculated by length×(width)²/2.

### Statistical Analysis:

Student's t-tests and one-way ANOVA were used to analyze in vitro data (GraphPad Prism, CA, USA); Two-way ANOVA with repeated measurements was used to analyze in vivo data (R3.4.3, The R Foundation); the survival of tumor-bearing mice was analyzed using log-rank tests (GraphPad Prism). All tests were two-tailed and P<0.05 was considered statistically significant.

### REFERENCES

[1] Borden EC: Interferons alpha and beta in cancer: therapeutic opportunities from new insights. Nat Rev Drug Discov 2019, 18:219-34.
[2] Mullard A: Restoring IL-2 to its cancer immunotherapy glory. Nat Rev Drug Discov 2021, 20:163-5.
[3] Waldmann T A. Cytokines in Cancer Immunotherapy. Cold Spring Harb Perspect Biol 2018, 10.
[4] Liu J Q, Zhu J, Hu A, Zhang A, Yang C, Yu J, Ghoshal K, Basu S, Bai X F: Is AAV-delivered IL-27 a potential immunotherapeutic for cancer? Am J Cancer Res 2020, 10:3565-74.
[5] Car B D, Eng V M, Schnyder B, LeHir M, Shakhov A N, Woerly G, Huang S, Aguet M, Anderson T D, Ryffel B: Role of interferon-gamma in interleukin 12-induced pathology in mice. Am J Pathol 1995, 147:1693-707.
[6] Ryffel B: Interleukin-12: role of interferon-gamma in IL-12 adverse effects. Clin Immunol Immunopathol 1997, 83:18-20.
[7] Colombo M P, Trinchieri G: Interleukin-12 in anti-tumor immunity and immunotherapy. Cytokine Growth Factor Rev 2002, 13:155-68.
[8] Del Vecchio M, Bajetta E, Canova S, Lotze M T, Wesa A, Parmiani G, Anichini A: Interleukin-12: biological properties and clinical application. Clinical cancer research: an official journal of the American Association for Cancer Research 2007, 13:4677-85.
[9] Hu J, Sun C, Bernatchez C, Xia X, Hwu P, Dotti G, Li S: T-cell Homing Therapy for Reducing Regulatory T Cells and Preserving Effector T-cell Function in Large Solid Tumors. Clin Cancer Res 2018, 24:2920-34.
[10] Zhao Q, Hu J, Mitra A, Cutrera J, Zhang W, Zhang Z, Yan J, Xia X, Mahadeo K M, Livingston J A, Gorlick R, Li S: Tumor-targeted IL-12 combined with tumor resection yields a survival-favorable immune profile. J Immunother Cancer 2019, 7:154.
[11] Kerkar S P, Muranski P, Kaiser A, Boni A, Sanchez-Perez L, Yu Z, Palmer D C, Reger R N, Borman Z A, Zhang L, Morgan R A, Gattinoni L, Rosenberg S A, Trinchieri G, Restifo N P: Tumor-specific CD8+ T cells expressing interleukin-12 eradicate established cancers in lymphodepleted hosts. Cancer Res 2010, 70:6725-34.
[12] Zhang L, Morgan R A, Beane J D, Zheng Z, Dudley M E, Kassim S H, Nahvi A V, Ngo L T, Sherry R M, Phan G Q, Hughes M S, Kammula U S, Feldman S A, Toomey M A, Kerkar S P, Restifo N P, Yang J C, Rosenberg S A: Tumor-infiltrating lymphocytes genetically engineered with an inducible gene encoding interleukin-12 for the immunotherapy of metastatic melanoma. Clin Cancer Res 2015, 21:2278-88.
[13] Mansurov A, Ishihara J, Hosseinchi P, Potin L, Marchell T M, Ishihara A, Williford J M, Alpar A T, Raczy M M, Gray L T, Swartz M A, Hubbell J A: Collagen-binding IL-12 enhances tumour inflammation and drives the complete remission of established immunologically cold mouse tumours. Nat Biomed Eng 2020, 4:531-43.
[14] Bhatia S, Longino N V, Miller N J, Kulikauskas R, Iyer J G, Ibrani D, Blom A, Byrd D R, Parvathaneni U, Twitty C G, Campbell J S, Le M H, Gargosky S, Pierce R H, Heller R, Daud A1, Nghiem P: Intratumoral Delivery of Plasmid IL12 Via Electroporation Leads to Regression of Injected and Noninjected Tumors in Merkel Cell Carcinoma. Clin Cancer Res 2020, 26:598-607.
[15] Hewitt S L, Bailey D, Zielinski J, Apte A, Musenge F, Karp R, Burke S, Garcon F, Mishra A, Gurumurthy S, Watkins A, Arnold K, Moynihan J, Clancy-Thompson E, Mulgrew K, Adjei G, Deschler K, Potz D, Moody G, Leinster D A, Novick S, Sulikowski M, Bagnall C, Martin P, Lapointe J M, Si H, Morehouse C, Sedic M, Wilkinson R W, Herbst R, Frederick J P, Luheshi N: Intratumoral IL12 mRNA Therapy Promotes TH1 Transformation of the Tumor Microenvironment. Clin Cancer Res 2020, 26:6284-98.
[16] Li Y, Su Z, Zhao W, Zhang X, Momin N, Zhang C, Wittrup K D, Dong Y, Irvine D J, Weiss R: Multifunctional oncolytic nanoparticles deliver self-replicating IL-12 RNA to eliminate established tumors and prime systemic immunity. Nature Cancer 2020, 1:882-93.
[17] Momin N, Mehta N K, Bennett N R, Ma L, Palmeri J R, Chinn M M, Lutz E A, Kang B, Irvine D J, Spranger S, Wittrup K D: Anchoring of intratumorally administered cytokines to collagen safely potentiates systemic cancer immunotherapy. Sci Transl Med 2019, 11.
[18] Dranoff G, Jaffee E, Lazenby A, Golumbek P, Levitsky H, Brose K, Jackson V, Hamada H, Pardoll D, Mulligan R C: Vaccination with irradiated tumor cells engineered to secrete murine granulocyte-macrophage colony-stimulating factor stimulates potent, specific, and long-lasting anti-tumor immunity. Proc Natl Acad Sci USA 1993, 90:353943.
[19] Armstrong C A, Botella R, Galloway T H, Murray N, Kramp J M, Song I S, Ansel J C: Antitumor effects of granulocyte-macrophage colony-stimulating factor production by melanoma cells. Cancer Res 1996, 56:2191-8.
[20] van Elsas A, Hurwitz A A, Allison J P: Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med 1999, 190:355-66.
[21] DranoffG: GM-CSF-based cancer vaccines. Immunol Rev 2002, 188:147-54.
[22] Hege K M, Jooss K, Pardoll D: GM-CSF gene-modified cancer cell immunotherapies: of mice and men. Int Rev Immunol 2006, 25:321-52.
[23] Kantoff P W, Higano C S, Shore N D, Berger E R, Small E J, Penson D F, Redfern C H, Ferrari A C, Dreicer R, Sims R B, Xu Y, Frohlich M W, Schellhammer P F, Investigators I S: Sipuleucel-T immunotherapy for castration-resistant prostate cancer. N Engl J Med 2010, 363:411-22.
[24] Bommareddy P K, Patel A, Hossain S, Kaufman H L. Talimogene Laherparepvec (T-VEC) and Other Oncolytic Viruses for the Treatment of Melanoma. Am J Clin Dermatol 2017, 18:1-15.
[25] Hodi F S, Lee S, McDermott D F, Rao U N, Butterfield L H, Tarhini A A, Leming P, Puzanov I, Shin D, Kirkwood J M: Ipilimumab plus sargramostim vs ipilimumab alone for treatment of metastatic melanoma: a randomized clinical trial. JAMA 2014, 312:1744-53.
[26] Luke J J, Donahue H, Nishino M, Giobbie-Hurder A, Davis M, Bailey N, Ott P A, Hodi F S: Single Institution Experience of Ipilimumab 3 mg/kg with Sargramostim (GM-CSF) in Metastatic Melanoma. Cancer Immunol Res 2015, 3:986-91.
[27] Natividad K D, Junankar S R, Mohd Redzwan N, Nair R, Wirasinha R C, King C, Brink R, Swarbrick A, Batten M: Interleukin-27 Signaling Promotes Immunity against Endogenously Arising Murine Tumors. PLoS One 2013, 8:e57469.
[28] Shinozaki Y, Wang S, Miyazaki Y, Miyazaki K, Yamada H, Yoshikai Y, Hara H, Yoshida H: Tumor-specific cytotoxic T cell generation and dendritic cell function are differentially regulated by interleukin 27 during development of anti-tumor immunity. Int J Cancer 2009, 124:1372-8.
[29] Curran M A, Geiger T L, Montalvo W, Kim M, Reiner S L, A I-Shamkhani A, Sun J C, Allison J P: Systemic 4-1B B activation induces a novel T cell phenotype driven by high expression of Eomesodermin. J Exp Med 2013, 210:743-55.
[30] Wei J, Xia S, Sun H, Zhang S, Wang J, Zhao H, Wu X, Chen X, Hao J, Zhou X, Zhu Z, Gao X, Gao J X, Wang P, Wu Z, Zhao L, Yin Z: Critical role of dendritic cell-derived IL-27 in antitumor immunity through regulating the recruitment and activation of N K and NKT cells.

Journal of immunology 2013, 191:500-8.
[31] Liu Z, Liu J Q, Talebian F, Wu L C, Li S, Bai X F: IL-27 enhances the survival of tumor antigen-specific CD8(+) T cells and programs them into IL-10-producing, memory precursor-like effector cells. Eur J Immunol 2013, 43:468-79.
[32] Hisada M, Kamiya S, Fujita K, Belladonna M L, Aoki T, Koyanagi Y, Mizuguchi J, Yoshimoto T: Potent antitumor activity of interleukin-27. Cancer Res 2004, 64:1152-6.
[33] Salcedo R, Stauffer J K, Lincoln E, Back T C, Hixon J A, Hahn C, Shafer-Weaver K, Malyguine A, Kastelein R, Wigginton J M: IL-27 mediates complete regression of orthotopic primary and metastatic murine neuroblastoma tumors: role for CD8+ T cells. Journal of immunology 2004, 173:7170-82.
[34] Liu Z, Liu J Q, Shi Y, Zhu X, Liu Z, Li M S, Yu J, Wu L C, He Y, Zhang G, Bai X F: Epstein-Barr virus-induced gene 3-deficiency leads to impaired antitumor T-cell responses and accelerated tumor growth. Oncoimmunology 2015, 4:e989137.
[35] Hu A, Ding M, Zhu J, Liu J Q, Pan X, Ghoshal K, Bai X F: Intra-Tumoral Delivery of IL-27 Using Adeno-Associated Virus Stimulates Anti-tumor Immunity and Enhances the Efficacy of Immunotherapy. Front Cell Dev Biol 2020, 8:210.
[36] Zhang C, Zhang X, Zhao W, Zeng C, Li W, Li B, Luo X, Li J, Jiang J, Deng B, McComb D W, Dong Y: Chemotherapy drugs derived nanoparticles encapsulating mRNA encoding tumor suppressor proteins to treat triple-negative breast cancer. Nano Res 2019, 12:855-61.
[37] Zhu S, Lee D A, Li S: IL-12 and IL-27 sequential gene therapy via intramuscular electroporation delivery for eliminating distal aggressive tumors. J Immunol 2010, 184:2348-54.
[38] Yoshimoto T, Chiba Y, Furusawa J, Xu M, Tsunoda R, Higuchi K, Mizoguchi I: Potential clinical application of interleukin-27 as an antitumor agent. Cancer Sci 2015, 106:1103-10.
[39] Zhu J, Liu J Q, Shi M, Cheng X, Ding M, Zhang J C, Davis J P, Varikuti S, Satoskar A R, Lu L, Pan X, Zheng P, Liu Y, Bai X F: IL-27 gene therapy induces depletion of Tregs and enhances the efficacy of cancer immunotherapy. JCI Insight 2018, 3.
[40] Morales J K, Kmieciak M, Knutson K L, Bear H D, Manjili M H: GM-CSF is one of the main breast tumor-derived soluble factors involved in the differentiation of CD1 lb-Grl-bone marrow progenitor cells into myeloid-derived suppressor cells. Breast Cancer Res Treat 2010, 123:39-49.
[41] Rosborough B R, Castellaneta A, Natarajan S, Thomson A W, Turnquist H R: Histone deacetylase inhibition facilitates GM-CSF-mediated expansion of myeloid-derived suppressor cells in vitro and in vivo. J Leukoc Biol 2012, 91:701-9.
[42] Zhang X, Zhao W, Nguyen G N, Zhang C, Zeng C, Yan J, Du S, Hou X, Li W, Jiang J, Deng B, McComb D W, Dorkin R, Shah A, Barrera L, Gregoire F, Singh M, Chen D, Sabatino D E, Dong Y: Functionalized lipid-like nanoparticles for in vivo mRNA delivery and base editing. Science Advances 2020, 6:eabc2315.
[43] Li B, Luo X, Deng B, Giancola J B, McComb D W, Schmittgen T D, Dong Y: Effects of local structural transformation of lipid-like compounds on delivery of messenger RNA. Scientific Reports 2016, 6:22137.

1. A composition comprising a compound defined by Formula I, or a pharmaceutically acceptable salt thereof: 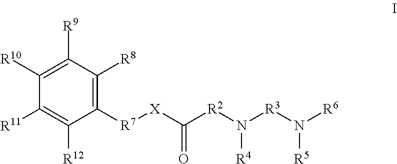
wherein
X is O, S, or NR¹;
R¹ is hydrogen, substituted or unsubstituted C₁-C₁₀ alkyl, or substituted or unsubstituted C₃-C₁₀ aryl group;
R² is substituted or unsubstituted C₁-C₁₀ alkyl, or substituted or unsubstituted C₁-C₁₀ alkenyl;
R³ is substituted or unsubstituted C₁-C₅ alkyl; and
R⁴, R⁵, and R⁶ are each independently substituted or unsubstituted C₆-C₂₀ alkyl;
R⁷ is substituted or unsubstituted C₁-C₅ alkyl;
R⁸, R⁹, R¹⁰, R¹¹, and R¹² are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ heteroaryl, substituted or unsubstituted C₃-C₂₀ cycloalkyl, substituted or unsubstituted C₁-C₂₀ acyl, or NR Y, or wherein, as valence permits, R⁸ and R⁹, R⁹ and R¹⁰, R¹⁰ and R¹¹, or R¹¹ and R¹², together with the atoms to which they are attached, form a 3-10 membered substituted or unsubstituted cyclic moiety optionally including from 1 to 3 heteroatoms; and
R^{x} and R^{y} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

2. The composition of claim 1, wherein R² is a substituted or unsubstituted C₄-C₆ alkyl.

3. (canceled)

4. (canceled)

5. (canceled)

6. The composition of claim 1, wherein R³ is a substituted or unsubstituted C₂-C₄ alkyl.

7. (canceled)

8. (canceled)

9. (canceled)

10. The composition of claim 1, wherein R⁴, R⁵, and R⁶ are each independently a substituted or unsubstituted C₁₀-C₁₄ alkyl.

11. (canceled)

12. (canceled)

13. The composition of claim 1, wherein R⁴, R⁵, and R⁶ are the same.

14. (canceled)

15. The composition of claim 1, wherein the compound is defined by Formula II: 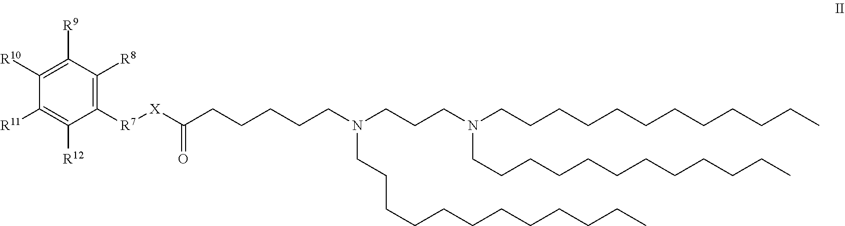
or a pharmaceutically acceptable salt thereof.

16. The composition of claim 1, wherein X is NR¹.

17. The composition of claim 1, wherein the compound is defined by Formula III: 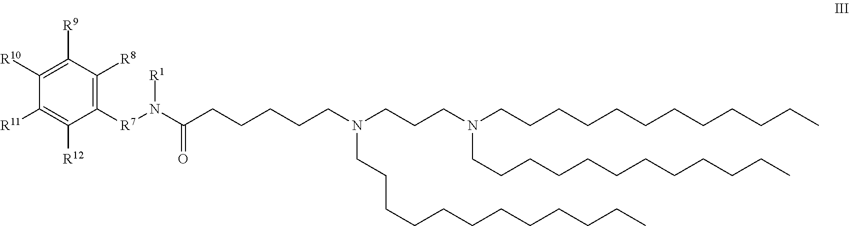
or a pharmaceutically acceptable salt thereof.

18. The composition of claim 17, wherein the compound is defined by Formula IV: 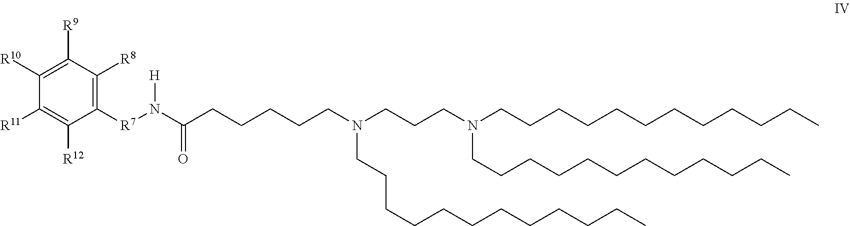
or a pharmaceutically acceptable salt thereof.

19. (canceled)

20. (canceled)

21. The composition of claim 17, wherein the compound is defined by Formula VII, or a pharmaceutically acceptable salt thereof: 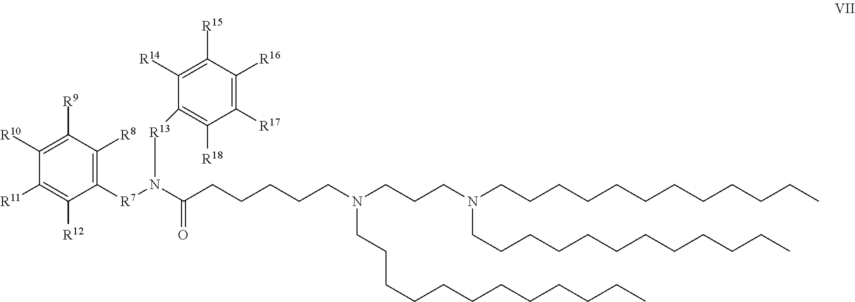
wherein
R¹³ is substituted or unsubstituted C₁-C₅ alkyl;
R¹⁴, R¹⁵, R¹⁶, R¹⁷, and R¹⁸ are each independently H, OH, halogen, boronic acid, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, substituted or unsubstituted C₁-C₂₀ acyl, or NR^{a}R^{b}, or wherein, as valence permits, R¹⁴ and R¹⁵, R¹⁵ and R¹⁶, R¹⁶ and R¹⁷, or R¹⁷ and R¹⁸, together with the atoms to which they are attached, form a 3-10 membered substituted or unsubstituted cyclic moiety optionally including from 1 to 3 heteroatoms; and
R^{a} and R^{b} are independently selected from substituted or unsubstituted C₁-C₂₀ alkyl, substituted or unsubstituted C₂-C₂₀ alkenyl, substituted or unsubstituted C₂-C₂₀ alkynyl, substituted or unsubstituted C₃-C₂₀ aryl, substituted or unsubstituted C₄-C₂₀ alkylaryl, substituted or unsubstituted C₃-C₂₀ cycloalkyl, substituted or unsubstituted C₃-C₂₀ heteroaryl, or substituted or unsubstituted C₁-C₂₀ acyl.

22. (canceled)

23. The composition of claim 1, wherein the compound is selected from the group consisting of: 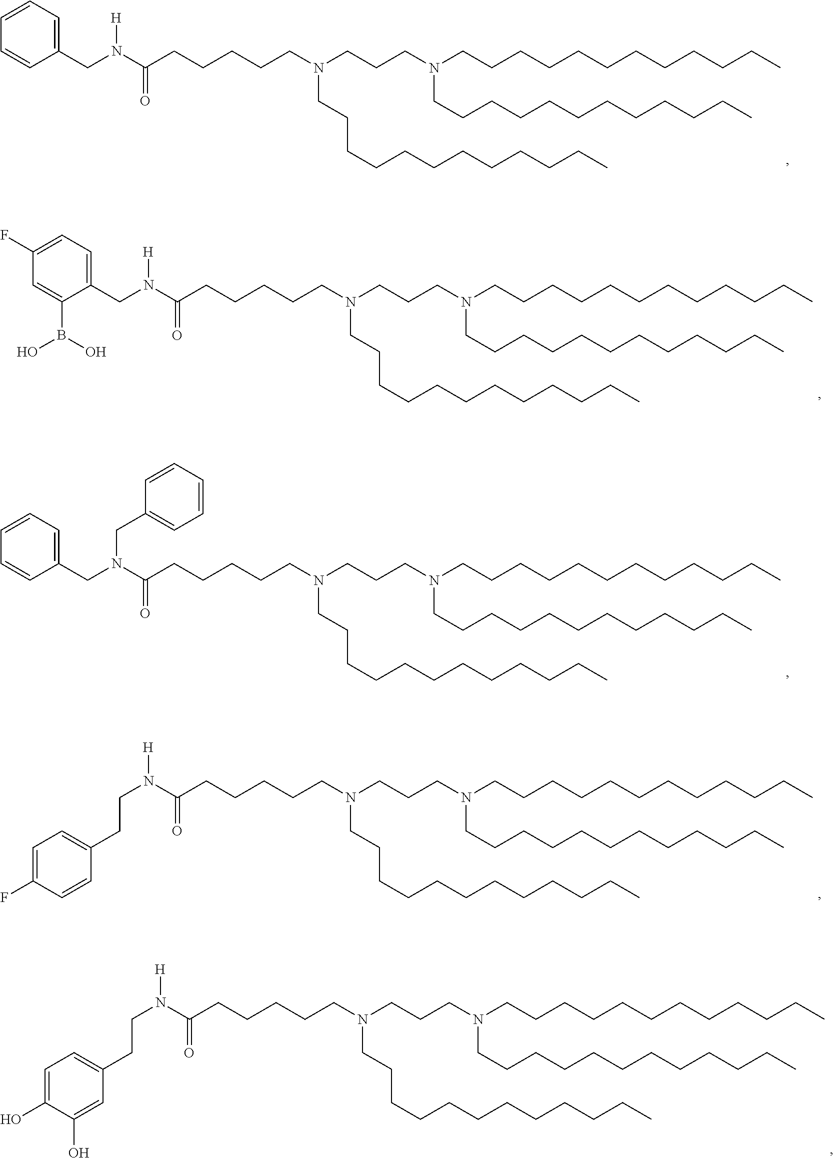 [SMILES: CCCCCCCCCCCCN(CCCCCCCCCCCC)CCCN(CCCCCCCCCCCC)CCCCCC(=O)NCc1ccccc1 ; CCCCCCCCCCCCN(CCCCCCCCCCCC)CCCN(CCCCCCCCCCCC)CCCCCC(=O)NCc1ccc(F)cc1B(O)O ; CCCCCCCCCCCCN(CCCCCCCCCCCC)CCCN(CCCCCCCCCCCC)CCCCCC(=O)N(Cc1ccccc1)Cc1ccccc1 ; CCCCCCCCCCCCN(CCCCCCCCCCCC)CCCN(CCCCCCCCCCCC)CCCCCC(=O)NCCc1ccc(F)cc1 ; CCCCCCCCCCCCN(CCCCCCCCCCCC)CCCN(CCCCCCCCCCCC)CCCCCC(=O)NCCc1ccc(O)c(O)c1]
or a pharmaceutically acceptable salt thereof.

24. (canceled)

25. (canceled)

26. A lipid particle comprising the composition of claim 1.

27. (canceled)

28. (canceled)

29. (canceled)

30. (canceled)

31. The lipid particle of claim 26, wherein the lipid particle further comprises an additional component.

32. (canceled)

33. The lipid particle of claim 31, wherein the additional component comprises a phospholipid, a sterol, or a combination thereof.

34. (canceled)

35. A pharmaceutical composition comprising a therapeutic agent encapsulated within the lipid particle of claim 26.

36. (canceled)

37. The pharmaceutical composition of claim 35, wherein the therapeutic agent comprises an anticancer agent, an anti-inflammatory agent, an antimicrobial agent, or a combination thereof.

38. (canceled)

39. The pharmaceutical composition of claim 35, wherein the therapeutic agent comprises a nucleic acid.

40. The pharmaceutical composition of claim 39, wherein the nucleic acid is mRNA.

41. The pharmaceutical composition of claim 40, wherein the mRNA encodes a cytokine.

42. (canceled)

43. (canceled)

44. (canceled)

45. (canceled)

46. (canceled)

47. A method of treating a disease or disorder in a subject in need thereof, the method comprising administering to the subject a therapeutically effective amount of the pharmaceutical composition of claim 35.

48. (canceled)

49. (canceled)
